(19) United States
(12) Patent Application Publication
(10) Pub. No.: US 2025/0153421 A1
(43) Pub. Date: May 15, 2025

(54) GRADIENT PRINTING RESERVOIR AND PRINTING METHOD

(71) Applicant: Amsilk GmbH, Neuried (DE)

(72) Inventors: Vanessa Neubauer, Bayreuth (DE); Fredrick Ugonna, Bayreuth (DE); Frank Rieg, Bayreuth (DE); Bettina Alber-Laukant, Bayreuth (DE); Claudia Kleinschrodt, Bayreuth (DE); Florian Huter, Neuhaus ad. Pegnitz (DE); Johannes Wittmann, Bayreuth (DE)

(21) Appl. No.: 18/834,760

(22) PCT Filed: Feb. 9, 2023
(86) PCT No.: PCT/EP2023/053157

§ 371 Date: Jul. 31, 2024

(30) Foreign Application Priority Data
Feb. 10, 2022 (EP) 22156230.9

(51) Int. Cl. B29C 64/106; B33Y 10/00; B33Y 70/10

(52) U.S. Cl. CPC B29C 64/106; B33Y 10/00; B33Y 70/10

(57) ABSTRACT

The present invention relates to a reservoir for gradient printing. The present invention further relates to a method of dispensing a content from a reservoir. The present invention also relates to different uses of the reservoir.

The present invention relates to a reservoir for gradient printing. The present invention further relates to a method of dispensing a content from a reservoir. The present invention also relates to different uses of the reservoir.

### BACKGROUND OF THE INVENTION

The range of applications for gradient printing such as 3D gradient printing becomes wider and wider. One promising form of 3D printing is bioprinting. In the future, biologically functioning tissue will be produced in the laboratory from biological material such as human cells. This offers a number of advantages. For example, patients with serious injuries could receive skin or muscle tissue from the lab. Cosmetic surgery could also benefit from the bioprinting process.

Bioprinting is an additive manufacturing process that combines biological material such as cells and growth factors. This material is used to create tissue-like structures that mimic natural tissue.

The technology uses a material called bioink. Similar to inkjet printing, the bioprinter prints layer by layer. As with tissue engineering, biological and biologically functional tissues are produced in the laboratory. A broad field of potential applications in medicine and bioengineering is opening up for this novel technology. The technology has already seen recent progress in the production of cartilage tissue for reconstruction and regeneration.

There are already first approaches for the production and use of in vitro tissues from the printer. As test systems, these bioprinted tissues can replace animal tests and provide information on the efficacy of drug candidates. In the future, animal testing could, thus, become unnecessary. In the foreseeable future, damaged tissue could be completely replaced by biological implants or stimulated to regenerate.

Specific hierarchical structures play an important role in almost all tissues of the human body. Therefore, it is essential for many tissue engineering approaches and, therefore, for biofabrication, to mimic such hierarchical structures in order to gain function of the new tissue. The hierarchical organization of tissue is essentially contributing its function. Hierarchies are seen on two levels, on a structural/morphological one and on a functional one. Mechanical gradient materials often mediate between two materials with different properties to gain smooth transitions and, therefore, to prevent material failure at their interface. This concept can be found, for example, at the tendon-bone-insertion, the so-called enthesis, with material gradients and resulting gradually changing mechanical properties.

Current engineering approaches for gradient design are focusing on 3D printing, as a versatile tool to generate predefined complex hierarchical structures. The simultaneous printing of two or more materials has so far been realized using co-printing approaches from separate cartridges resulting in multilayer constructs or concentration patterns or from combined cartridges with a mixing unit. In the combined cartridges, the materials are mixed and extruded in a gradual manner via an outlet. For this purpose, the material flow of the two cartridges has to be controlled separately. The installation of a Y-connector (also known as a T-mixer) requires financial investment and is accompanied by modifications to the unit (if at all possible), which may then no longer be available for all applications.

Presently, there is no printing system available in which an in situ gradient can be generated during dispensing pressure with only one filled cartridge without further aids. In particular, no printing method presently exists using a single cartridge without further tools or additional equipment which is able to print gradients independently of available equipment. Accordingly, there is a strong need for new gradient printing systems and methods using said systems.

The present inventors generated for the first time an in situ gradient of different mechanical properties or different functions (e.g. antibiotic gradient, growth factor gradient, etc.) during dispensing pressure with a filled cartridge. Specifically, the present inventors fed two individual material blocks into a cartridge and printed them. During printing, a material gradient was created from the two materials within the cartridge. By filling the cartridge in a delineated block system, the materials were initially not mixed and exhibited an interface. By applying pressure, both materials were extruded simultaneously. The laminar flow during extrusion provided a U-profile and, thus, created a gradual mixing of the materials, which were then extruded in a gradient manner.

A modification of 3D printers is, thus, not necessary anymore. The cartridge developed by the present inventors can rather be used or inserted in existing printer systems. This saves costs and simplifies handling.

In a specific example, the present inventors focused on the so-called enthesis which is a gradient structure, located between tendon and bone, bridging their mechanical properties to prevent crack propagation. Especially, recombinant silk hydrogels were combined with fluorapatite rods, and a mineralized gradient was generated using two hydrogels, one with and one without fluorapatite in a dispense plotting set-up. Finally, fluorapatite particles and BALB/3T3 fibroblasts were encapsulated in one recombinant silk hydrogel and gradually printed with the blank hydrogel.

With such set-ups, adjustable gradient features can be achieved, useful for tissue engineering application, e.g. at the tendon/bone interface and elsewhere. The technique of the present inventors is of interest for various other applications, e.g. in the pharmaceutical industry, in the field of tissue reconstruction, medical technology, cell cultivation, or for test arrays.

### SUMMARY OF THE INVENTION

In a first aspect, the present invention relates to a reservoir comprising
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
a first flowable composition comprising a first material, and
a second flowable composition comprising a second material.

In a preferred embodiment, the reservoir is a cartridge.

In a second aspect, the present invention relates to a printer comprising the cartridge according to the first aspect or a receptacle for such a cartridge.

In a third aspect, the present invention relates to a method of dispensing a content from a reservoir comprising the step of:
applying pressure to a content of the reservoir, so that the content is dispensed from the reservoir via an outlet, wherein the content comprises
a first flowable composition comprising a first material, and
a second flowable composition comprising a second material.

In a fourth aspect, the present invention relates to a kit comprising the reservoir, preferably cartridge, according to the first aspect or the printer according to the second aspect.

In a fifth aspect, the present invention relates to the use of the reservoir, preferably cartridge, according to the first aspect or the printer according to the second aspect for bioprinting.

In a sixth aspect, the present invention relates to the use of the reservoir, preferably cartridge, according to the first aspect or the printer according to the second aspect for biofabrication.

In a seventh aspect, the present invention relates to the use of the reservoir, preferably cartridge, according to the first aspect or the printer according to the second aspect for tissue engineering applications.

In an eighth aspect, the present invention relates to the use of the reservoir, preferably cartridge, according to the first aspect or the printer according to the second aspect for gradient formation.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments. However, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The term “biomaterial”, as used herein, refers to a material that has been engineered to interact with biological systems for a medical purpose, either a therapeutic (treat, augment, repair, or replace a tissue function of the body) or a diagnostic one. The term “biomaterial”, as used herein, further refers to a material which has been adapted and used for printing such as gradient printing, e.g. of three-dimensional objects. Some of the most notable bioengineered substances are usually stronger than the average bodily materials, including soft tissue and bone. These constituents can act as future substitutes, even improvements, for the original body materials. Silk polypeptides are suitable as biomaterials. Alginate, for example, is an anionic polymer with many biomedical implications including feasibility, strong biocompatibility, low toxicity, and stronger structural ability in comparison to some of the body's structural material. Preferably, the biomaterial is a biopolymer. More preferably, the biopolymer is selected from the group consisting of a silk polypeptide, alginate, collagen, cellulose, and chitosan. Even more preferably, the biopolymer is a silk polypeptide.

The terms “polypeptide” and “protein” are used interchangeably in the context of the present invention. They refer to a long peptide-linked chain of amino acids, e.g. one that is at least 40 amino acids long.

The term “silk polypeptide”, as used herein, refers to a polypeptide which comprises repetitive units/repeating building blocks made of amino acids. Specifically, a silk polypeptide possesses large quantities of hydrophobic amino acids such as glycine or alanine. Especially, the highly repetitive amino acid sequences are located in the large core domain of the silk polypeptide.

Based on DNA analysis, it was shown that all silk polypeptides comprise chains of repetitive units which further comprise a limited set of distinct shorter peptide motifs. The expressions “peptide motif” and “consensus sequence” can be used interchangeably herein. Generally, the silk consensus sequences can be grouped into four major categories: GPGXX, GGX, Aₓ or (GA)ₙ and spacers. These categories of peptide motifs in silk proteins have been assigned structural roles. For example, it has been suggested that the GPGXX motif is involved in a β-turn spiral, probably providing elasticity. The GGX motif is known to be responsible for a glycine-rich 31-helix. Both GPGXX and GGX motifs are thought to be involved in the formation of an amorphous matrix that connects crystalline regions, thereby providing elasticity of the fiber. Alanine-rich motifs typically contain 6 to 9 residues and have been found to form crystalline β-sheets. The spacers typically contain charged groups and separate the iterated peptide motifs into clusters.

The silk polypeptide can perform self-assembly. Preferably, the silk polypeptide is a recombinant silk polypeptide. More preferably, the silk polypeptide is a spider silk polypeptide. Even more preferably, the spider silk polypeptide is a recombinant spider silk polypeptide.

An exemplarily process for producing a silk polypeptide used herein is described in WO 2006/008163 and in WO 2011/120690.

The term “self-assembly”, as used herein, refers to a process in which a disordered system of pre-existing polypeptides forms an organized structure or pattern as a consequence of specific, local interactions (e.g. van der Waals forces, hydrophobic interactions, hydrogen bonds, and/or salt-bridges, etc.) among the polypeptides themselves, without external direction or trigger although external factors might influence speed and nature of self-assembly. This particularly means that when two or more disordered and/or unfolded polypeptides are brought into contact, they interact with each other and consequently form a three-dimensional structure. The change from a disordered system to an organized structure or pattern during self-assembly is characterized by a transition from a fluid state to a gelatinous/gel-like and/or solid state and a corresponding increase in viscosity. The transition from a fluid state to a gelatinous/gel-like state can be monitored, for example, by optical measurement or rheology. These techniques are known to the skilled person. The transition from a fluid state to a solid state can be monitored, for example, using optical methods. The polypeptides mentioned above are preferably silk polypeptides.

The term “polypeptide aggregates”, as used herein, refers to polypeptide structures which are formed as a consequence or result of polypeptide self-assembly. In the process of polypeptide self-assembly, multiple copies/units of polypeptides self-aggregate into a body or mass without external direction or trigger although external factors might influence speed and nature of self-assembly. In the polypeptide aggregates, the different polypeptides are connected with or attached to each other via covalent (e.g. disulfide bridges) and/or non-covalent interactions (e.g. van der Waals forces, hydrophobic interactions, hydrogen bonds, and/or salt-bridges). It should be clear that a polypeptide aggregate encompasses at least two polypeptides. The polypeptide aggregates mentioned above are preferably silk polypeptides.

The term “composition”, as used herein, refers to a composition comprising a first material or a second material. The composition comprising a first material may further comprise one or more additives. In addition, the composition comprising a second material may further comprise one or more additives. The composition comprising the first material (and optionally one or more additives) is also designated as first composition and the composition comprising the second material (and optionally one or more additives) is also designated as second composition. Particularly, the first composition and the second composition differ from each other. Specifically, the first and the second composition differ from each other either in terms of the first and second material or in terms of the one or more additives comprised therein.

In one example, the first and second material is identical. In this case, the difference between the first and second composition lies within the one or more additives comprised therein.

In one another example, the first and second material is different. In this case, the difference between the first and second composition lies within the first and second material comprised therein. Optionally, one or more additives are part of the first and/or second composition. In case that one or more additives are part of the first and second composition, the one or more additives can be identical or different.

In one embodiment, the first and/or second material comprises a biomaterial. The biomaterial such as biopolymer is preferably selected from the group consisting of a silk polypeptide, alginate, gelatin, gelatin with methacrylated groups (GelMA), collagen, cellulose, and chitosan, or is a combination thereof. In one preferred embodiment, the first and/or second material comprises a silk polypeptide.

The difference between the first material and the second material lies in particular in the type, structure, concentration, and/or viscosity of the first material and the second material.

The term “flowable composition”, as used herein, refers to a composition that is able/capable of flowing or being flowed. A flowable composition is (still) in a liquid state. Described herein is a first flowable composition and a second flowable composition. In one preferred embodiment, the first and/or second flowable composition comprises a silk polypeptide. Said silk polypeptide is part of the first and/or second flowable composition as first and/or second material.

The followability of the flowable composition can easily be determined by the skilled person, e.g. by rheology or viscosity measurements. The followability measurements are preferably preformed under standard conditions (25° C.). Preferably, the (first and/or second) composition is a hydrogel.

The term “hydrogel”, as used herein, refers to a structure that is formed if the concentration of liquid component comprised therein is high enough to build a continuous network by which the liquid component is immobilized. Said network is preferably formed by self-assembling of the liquid component providing the basis of the hydrogel. In particular, the hydrogel is a hydrophilic polymeric network of components. Said network is stabilized by chemical and/or physical interactions between the components. The network is dispersed throughout an immobilized aqueous phase.

In one embodiment, the hydrogel comprises a biomaterial. The biomaterial such as biopolymer is preferably selected from the group consisting of a silk polypeptide, alginate, gelatin, gelatin with methacrylated groups (GelMA), collagen, cellulose, and chitosan, or is a combination thereof. In one preferred embodiment, the hydrogel is a silk polypeptide hydrogel.

The term “flowable hydrogel”, as used herein, refers to a hydrogel that is able/capable of flowing or being flowed. A flowable hydrogel is (still) in a liquid state.

In one embodiment, the flowable hydrogel comprises a biomaterial. The biomaterial such as biopolymer is preferably selected from the group consisting of a silk polypeptide, alginate, gelatin, gelatin with methacrylated groups (GelMA), collagen, cellulose, and chitosan, or is a combination thereof. In one preferred embodiment, the flowable hydrogel is a silk polypeptide hydrogel.

The term “silk polypeptide hydrogel”, as used herein, refers to a structure that is formed if the concentration of silk polypeptides is high enough to build a continuous network by which the silk polypeptides are immobilized. Said network is preferably formed by self-assembling of the silk polypeptides providing the basis of the silk hydrogel. In particular, the hydrogel is a hydrophilic polymeric network of silk polypeptides. Said network is stabilized by chemical and/or physical interactions between the silk polypeptides. The network is dispersed throughout an immobilized aqueous phase. The hydrophilicity and stability of the hydrogel permits the penetration and absorption of water (swelling) without dissolving, thus, maintaining its three-dimensional (3D) structure and function. In one preferred embodiment, the silk polypeptide hydrogel is a flowable silk polypeptide hydrogel.

In particular, the (flowable) hydrogel, as described above, has a fibrillary structure or comprises fibrillary complexes. The presence of fibrillary complexes improves and simplifies the printing of the (flowable) hydrogel. The fibrillary complexes may have the form of net structures.

The term “additive”, as used herein, refers to any compound having a purpose that may be useful in the present invention, e.g. any compound that can be added to the composition filled into the reservoir disclosed herein and that can finally be printed. Preferably, the additive is a bioactive compound such as a pharmaceutical active compound/therapeutic agent. The term “bioactive compound”, as used herein, refers to any physical, chemical, or biological substance which may be used in the treatment, cure, prophylaxis, or prevention of a pathological condition, e.g. a disease or disorder, or which may be used to otherwise enhance physical, psychical or mental well-being. Accordingly, the term “pharmaceutically active compound”, as used herein, includes any compound with therapeutic, curative, prophylactic, or preventative effect. The terms “pharmaceutically active compound” and “drug” can interchangeably be used herein. For example, the compound can be a compound that affects or participates in tissue growth, cell growth, cell differentiation, a compound that is able to invoke a biological action such as an immune response, or a compound that can play any other role in one or more biological processes.

Preferably, the additive, in particular bioactive compound, is selected from the group consisting of an inorganic filler, a mineral, a cell, a virus, a spore, a dye, a protein, a lipid, a pharmaceutically active compound/drug, activated carbon, and cell culture material. More preferably, the additive, in particular bioactive compound, is a pharmaceutically active compound/drug such as a growth factor.

The term “growth factor”, as used herein, refers to a compound capable of stimulating cellular growth, proliferation and cellular differentiation. Typically, this compound is a protein or a steroid hormone. A growth factor is important for regulating a variety of cellular processes. It typically acts as signaling molecule between cells. An example of a growth factor is a cytokine or hormone that binds to specific receptors on the surface of their target cells. Specific examples of suitable growth factors include, but are not limited to, bone morphogenetic proteins (BMPs), epidermal growth factors (EGF), erythropoietin (EPO), fibroblast growth factor (FG), granulocyte-colony stimulating factor (G-CSF), granulocyte-macrophage stimulating factor (GM-CSF), growth differentiation factor-9 (GDF9), hepatocyte growth factor (HGF), hepatoma dervived growth factor (HDGF), insulin-like growth factor (HDGF), insulin-like growth factor (IGF), myostatin (GDF8), nerve-growth factor (NGF), platelet-dervived growth factor (PDGF), transforming growth factor alpha (TGF-α), transforming growth factor beta (TGF-β), and vascular ecdothelial growth factor (VEGF).

In one embodiment, the additive, in particular the bioactive compound, is encapsulated in silk polypeptide capsules (as described in EP1757276). Specifically, the additive, in particular the bioactive compound, is comprised in the silk polypeptide capsules, e.g. in the inner lumen and/or matrix of the silk capsules, and/or attached to the silk polypeptide capsules, e.g. the shell of the silk capsules.

In one another embodiment, the additive, in particular the bioactive compound, is coated with silk polypeptides. In the examples, fluorapatite particles coated with the silk polypeptide C₁₆ in which all glutamic acid residues are replaced by lysine ones (κ16) were incorporated into a silk polypeptide C₁₆ hydrogel for printing (first flowable composition). Alternatively, cells as well as fluorapatite particles coated with the silk polypeptide C₁₆ in which all glutamic acid residues are replaced by lysine ones (κ16) were incorporated into a silk polypeptide C₁₆ hydrogel for printing (first flowable composition). Additionally, a silk polypeptide C₁₆ hydrogel was used (second flowable composition).

The term “coating”, as used herein, refers to a covering that is applied to a surface of an object. In particular, the term “coating”, as used herein, refers to a covering that is applied to the surface of the additive, in particular the bioactive compound, to be coated. The coating itself may be an all-over coating, completely covering the additive, in particular the bioactive compound, or it may only cover parts of the additive, in particular the bioactive compound. The coating may be a film or gel, in particular hydrogel. For example, the silk polypeptides used for the coating can be in form of an aerosol, a liquid, a gel, a paste, a semi-solid, or a solid. The coating is preferably achieved by dipping the additive into a silk polypeptide solution, spraying a silk polypeptide solution onto the surface of the additive, or nebulizing a silk polypeptide solution onto the surface of the additive.

The term “bioprinting”, as used herein, refers to an additive manufacturing process that combines biomaterials, optionally biomaterials and one or more additives, such as cells and/or growth factors. These biomaterials, optionally biomaterials and one or more additives, such as cells and/or growth factors, can be used to create tissue-like structures that mimic natural tissue. The bioprinting technology uses a material called bioink. Similar to inkjet printing, the bioprinter prints layer by layer. As with tissue engineering, biological and biologically functional tissues are produced in the laboratory. A broad field of potential applications in medicine and bioengineering is opening up for this novel technology. The technology has already seen recent progress in the production of cartilage tissue for reconstruction and regeneration.

The term “Three dimensional (3D) bioprinting”, as used herein, refers to the utilization of 3D printing like techniques to combine biomaterials, optionally biomaterials and one or more additives, such as cells and/or growth, to fabricate biomedical parts, e.g. to imitate natural tissue characteristics. Generally, 3D bioprinting can utilize a layer-by-layer method to deposit materials known as bioinks to create tissue-like structures that can later be used in various medical and tissue engineering fields. 3D bioprinting covers a broad range of bioprinting techniques.

For example, extrusion-based printing is a very common technique within the field of 3D printing which entails extruding, or forcing, a continuous stream of melted solid material or viscous liquid through a sort of orifice, often a nozzle or syringe. When it comes to extrusion based bioprinting, there are three main types of extrusion. These are pneumatic driven, piston driven, and screw driven. Each extrusion methods have their own advantages and disadvantages. Pneumatic extrusion used pressurized air to force liquid bioink through a depositing agent. The air used to move the bioink must be free of contaminants. Air filters are commonly used to sterilize the air before it is used. Piston driven extrusion utilizes a piston connected to a guide screw. The linear motion of the piston squeezes material out of the nozzle. Screw driven extrusion uses an auger screw to extrude material. The rotational motion forces the material down and out of the nozzle. Screw driven devices allow for the use of higher viscosity materials and provide more volumetric control.

Direct extrusion is one of the most common extrusion-based bioprinting techniques, wherein the pressurized force directs the bioink to flow out of the nozzle, and directly print the scaffold without any necessary casting. The bioink itself for this approach can be a blend of polymer hydrogels, e.g. naturally derived materials such as silk polypeptides and live cells suspended in the solution. In this manner, scaffolds can be cultured post-print and not need further treatment for cellular seeding. Indirect extrusion techniques for bioprinting rather require the printing of a base material of cell-laden hydrogels, but unlike direct extrusion contains a sacrificial hydrogel that can be trivially removed post-printing through thermal or chemical extraction. The remaining material solidifies and becomes the desired 3D-printed construct.

The term “bioink”, as used herein, refers to a biomaterial which is printable. Bioinks are materials which can be used to produce engineered/artificial live tissue. These inks are biomaterial based and are often further composed of cells. The cells are sometimes used in tandem with additional material that envelopes/coats the cells. This additional material can be the biomaterial itself. The bioink must meet certain characteristics, including such as rheology, mechanical, biofunctional and biocompatibility properties, among others. Preferably, the biomaterial is a biopolymer. More preferably, the biopolymer is selected from the group consisting of a silk polypeptide, alginate, gelatin, gelatin with methacrylated groups (GelMA), collagen, cellulose, and chitosan, or is a combination thereof. Specifically, the bioink comprises a silk polypeptide. Particularly, the bioink comprises a first flowable composition and a second flowable composition, wherein the first flowable composition and/or second flowable composition comprises a silk polypeptide (as first and/or second material).

The term “biofabrication”, as used herein, refers to the development and cultivation of bioartificial, cellular tissues using a variety of biotechnological, biomedical, and materials science and engineering methods, processes, and procedures. The present inventors used bioprinting, in particular 3D bioprinting, to fabricate a bioproduct.

In this respect, it should be noted that 3D bioprinting is an additive manufacturing process for the production of living tissue. For example, hydrogels loaded with living cells are printed layer-by-layer to generate a precursor structure that can mature into functional, biological tissue in subsequent culturing steps. Compared to other biofabrication methods, such as cell colonization or molding, 3D bioprinting has several advantages that are important for the production of complex tissues. These include the spatially controlled and reproducible placement of different biomaterials and cells in a biological structure, as well as the production of patient-specific 3D geometries. Another advantage is the high degree of automation and digitization of the process.

The term “tissue engineering”, as used herein, refers to a biomedical engineering discipline that uses a combination of cells, engineering, materials methods, and suitable biochemical and physicochemical factors to restore, maintain, improve, or replace different types of biological tissues. Tissue engineering often involves the use of cells placed on tissue scaffolds in the formation of new viable tissue for a medical purpose but is not limited to applications involving cells and tissue scaffolds.

The term “gradient printing”, as used herein, refers to a technique of distributing a composition so that the concentration or level of different biomaterials and/or additives comprised therein continuously changes across an area. These concentrations or gradients are specifically important for recapitulating native gradients of signalling molecules as well as varying levels of matrix stiffness that occurs in tissues. For example, various planar and 3D structures exhibiting continual gradients of materials, of cell densities, of growth factor concentrations, of hydrogel stiffness, and of porosities in horizontal and/or vertical direction, are possible. The composable fabrication of multifunctional gradients strongly supports the potential of the unique bioprinting system in numerous biomedical applications.

### Embodiments of the Invention

The present inventors generated for the first time an in situ gradient of different mechanical properties or different functions (e.g. antibiotic gradient, growth factor gradient, etc.) during dispensing pressure with a filled cartridge. Specifically, the present inventors fed two individual material blocks into a cartridge and printed them. During printing, a material gradient was created from the two materials within the cartridge. By filling the cartridge in a delineated block system, the materials were initially not mixed and exhibited an interface. By applying pressure, both materials were extruded simultaneously. The laminar flow during extrusion provided a U-profile and, thus, created a gradual mixing of the materials, which were then extruded in a gradient manner.

A modification of 3D printers is, thus, not necessary anymore. The cartridge developed by the present inventors can rather be used or inserted in existing printer systems. This saves costs and simplifies handling.

Thus, in a first aspect, the present invention relates to a reservoir comprising
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
a first flowable composition comprising a first material, and
a second flowable composition comprising a second material.

In at least one embodiment, the reservoir is configured such that during a dispensing operation for dispensing content from the reservoir, at least a part of the dispensed content comprises at the outlet the first flowable composition and the second flowable composition which leave the reservoir simultaneously through the outlet.

In at least one embodiment, the first flowable composition and the second flowable composition which leave the reservoir simultaneously through the outlet can be deposited simultaneously on a target surface. The target surface may be a (polystyrene) dish, e.g. petri dish, or a plane surface.

In at least one embodiment, the first flowable composition and the second flowable composition have a common boundary surface between them.

The common boundary surface may extend across the entire diameter of the interior space. The common boundary surface may further divide the interior space into two portions, a first portion with the first flowable composition, particularly free of the second flowable composition, and a second portion with the second flowable composition, particularly free of the first flowable composition.

In at least one particular embodiment, the common boundary surface is arranged within the interior space. In at least one another particular embodiment, the reservoir is configured such that the common boundary surface comprises a plane central region when the content is not pressurised, or the reservoir is configured such that the common boundary surface comprises a non-plane central region when the content is pressurised.

In at least one embodiment, the reservoir is configured such that pressurisation of the content results in a non-plane boundary surface between the first flowable composition and the second flowable composition.

In at least one particular embodiment, the non-plane boundary surface comprises a concave central region as seen along a dispensing direction towards the outlet. In at least one another particular embodiment, the non-plane boundary surface has a U-shaped cross section.

In at least one embodiment, the reservoir body reduces its inner cross section or tapers in a transition portion of the reservoir body as seen from the interior space towards the outlet along the flow direction from the interior space to the outlet. For example, the reservoir body reduces its inner cross section e.g. taken perpendicular to a main longitudinal axis of the reservoir body. In at least one particular embodiment, the transition portion is curved. In at least one another particular embodiment, the transition portion comprises an inner surface with a concavely curved region and a convexly curved region, as seen from within the reservoir body onto the inner surface. In at least one specific embodiment, the concavely curved portion is further away from the outlet or closer to the interior space than the convexly curved portion. In at least one another specific embodiment, the inner cross section is greater in the concavely curved portion than in the convexly curved portion.

In at least one embodiment, an end portion of the reservoir or of the reservoir body is arranged between the outlet and the transition region.

In at least one particular embodiment, an end of the end portion remote from the interior space defines the outlet. In at least one another particular embodiment, an inner cross section of the end portion decreases, preferably continuously and/or monotonously, in the direction towards the outlet and/or away from the interior space. In at least one specific embodiment, the inner cross section of the end portion is conical.

In at least one embodiment, the interior space is continuous and/or has a constant cross-section.

In at least one embodiment, the reservoir is configured to dispense the content from the reservoir through the outlet with a laminar flow.

In at least one embodiment, the reservoir is configured to dispense the content from the interior space through the outlet such that the dispensed content can exhibit a gradient or change in the proportion of the first flowable composition and the proportion of the second flowable composition in the dispensed content.

For example, the dispensed content can exhibit a gradient or change in the proportion of the first flowable composition and the proportion of the second flowable composition in the dispensed content as seen along a dispensing direction which extends along the dispensed content and away from the outlet.

In at least one particular embodiment, the gradient changes along the dispensing direction or is constant. In at least one another particular embodiment, the dispensed content comprises a region with a boundary between the first flowable composition and the second flowable composition. In one specific embodiment, the dispensed content comprises a first region consisting of the first flowable composition. Alternatively, the dispensed content comprises a second region consisting of the second flowable composition. Alternatively, the dispensed content comprises a combined region comprising both, the first flowable composition and the second flowable composition.

Particularly, a proportion of the first flowable composition in the dispensed content increases within the combined region as seen from the combined region in a direction towards the first region and/or as seen from the combined region in a direction away from the second region along the dispensed content, and/or a proportion of the second flowable composition in the dispensed content decreases within the same ratio as the first flowable composition increases within the combined region as seen from the combined region in a direction towards the first region and/or as seen from the combined region in a direction away from the second region along the dispensed content.

Alternatively or more particularly, a proportion of the second flowable composition in the dispensed content increases within the combined region as seen from the combined region in a direction towards the second region and/or as seen from the combined region in a direction away from the first region along the dispensed content, and/or a proportion of the first flowable composition in the dispensed content decreases within the same ratio as the second flowable composition increases within the combined region as seen from the combined region in a direction towards the second region and/or as seen from the combined region in a direction away from the first region along the dispensed content.

As mentioned above, in at least one embodiment, the dispensed content comprises a region with a boundary between the first flowable composition and the second flowable composition. For example, the boundary between the first flowable composition and the second flowable composition in the dispensed content is oblique relative to the dispensing direction.

In at least one embodiment, the interior space is circumferentially delimited by a cylindrical surface of the reservoir body.

In at least one embodiment, the reservoir body has an opening. In at least one particular embodiment, the opening is at an end of the reservoir body remote from the outlet.

In at least one embodiment, the interior space is delimited by a piston which is movably retained in the reservoir body.

In at least one particular embodiment, the piston seals the interior space of the reservoir as seen from the opening.

In at least one another particular embodiment, the piston has a non-plane contact portion for contacting the content to pressurise the content.

In at least one specific embodiment, the contact portion reduces its cross section or tapers as seen in a direction away from the opening. In at least one specific embodiment, the contact portion is conical.

In at least one particular embodiment, the piston is movable relative to the reservoir body, in particular towards the outlet, to pressurise the content.

In at least one another particular embodiment, one of the first flowable composition and the second flowable composition is arranged between the piston and the other one of the first flowable composition and the second flowable composition.

In the examples, a conical piston and a 14, 16, or 20 G needle was used. In addition, reservoirs with an inner diameter of 14, 16, or 20 G were used.

In at least one specific embodiment, the other one of the first flowable composition and the second flowable composition is arranged between the outlet and the one of the first flowable composition and the second flowable composition.

In at least one embodiment, the first flowable composition and/or the first material is a non-Newtonian fluid.

In at least one embodiment, the second flowable composition and/or the second material is a non-Newtonian fluid.

In at least one particular embodiment, the first flowable composition and/or the first material is a non-Newtonian fluid and the second flowable composition and/or the second material is a non-Newtonian fluid.

In at least one embodiment, the reservoir is a syringe. In this embodiment, the content comprised therein can be removed from/squeezed out the syringe by hand/manually, e.g. by driving the piston comprised therein relative to the syringe body. The syringe is suitable for application of a dermal filler. Thus, in a preferred embodiment, the syringe comprises a dermal filler which can be applied. In a more preferred embodiment, the syringe comprises a first dermal filler as first flowable composition and a second dermal filler as second flowable composition, wherein the first and second flowable composition differ from each other.

In at least one embodiment, the reservoir comprises a luer connector.

In at least one embodiment, the outlet is configured to be releasably coupled to a needle or nozzle or wherein the outlet is formed by an end of a needle or nozzle.

In at least one embodiment, the reservoir is configured to be connected to a pneumatic circuit/motor, a hydraulic circuit/motor, or electric circuit/motor to pneumatically, hydraulically, or electronically drive the piston relative to the reservoir body.

In at least one embodiment, the reservoir comprises a pneumatic or hydraulic connector for operatively connecting the reservoir to a pneumatic or hydraulic pressurisation device. In at least one particular embodiment, the pneumatic pressurisation or hydraulic device is configured to pressurise the piston pneumatically or hydraulically, with the piston transferring the pressure to the content.

In at least one embodiment, the reservoir is configured to be connected to an electric motor/transmission to electrically drive the piston relative to the reservoir body.

In at least one embodiment, the reservoir comprises a electric connector for operatively connecting the reservoir to an electric pressurisation device. In at least one particular embodiment, the electric pressurisation device is configured to pressurise the piston pneumatically or hydraulically, with the piston transferring the pressure to the content.

In at least one embodiment, the first flowable composition and the second flowable composition differ from each other. The difference between the first flowable composition and the second flowable composition may lie in the type, structure, concentration, and/or viscosity of the first flowable composition and second flowable composition. For example, the first flowable composition and the second flowable composition may differ in their mechanical properties. Specifically, the first flowable composition may be a stiff composition and the second flowable composition may be a soft composition, or vice versa. Alternatively, the first flowable composition may be a composition between soft and stiff (i.e. medium) and the second flowable composition may be a soft composition, or vice versa. Alternatively, the first flowable composition may be a composition between soft and stiff (i.e. medium) and the second flowable composition may be a stiff composition, or vice versa. By printing such compositions, a gradient of stiffness or softness can be reached. Alternatively, by printing such compositions graded stiffness from soft to medium or medium to soft as well as from stiff to medium or medium to stiff can be reached.

For simultaneous printing of the first flowable composition and the second flowable composition, it is preferred that the first and second materials comprised therein have flow points in the same order of magnitude.

In at least one preferred embodiment, the first flowable composition and/or the second flowable composition comprise one or more additives. Thus, the first flowable composition comprises, in addition to a first material, one or more additives and/or the second flowable composition comprises, in addition to a second material, one or more additives.

As mentioned above, the first flowable composition and the second flowable composition differ from each other. Thus, the first flowable composition and the second flowable composition may differ from each other in terms of the first and second material or in terms of the one or more additives comprised therein.

In one example, the first and second material is identical. In this case, the difference between the first flowable composition and second flowable composition lies within the one or more additives comprised therein.

In one another example, the first and second material is different. In this case, the difference between the first flowable composition and/or second flowable composition lies within the first and second material comprised therein. Optionally, one or more additives are comprised in the first flowable composition and/or second flowable composition. In case that one or more additives are part of the first flowable composition and/or second flowable composition, the one or more additives can be identical or different.

The difference between the first material and the second material may lie in the type, structure, concentration, and/or viscosity of the first material and the second material.

For example, the first material and the second material may differ in their mechanical properties. Specifically, the first material may be a stiff material and the second material may be a soft material, or vice versa. Alternatively, the first material may be a material between soft and stiff (i.e. medium) and the second material may be a soft material, or vice versa. Alternatively, the first material may be a material between soft and stiff (i.e. medium) and the second material may be a stiff material, or vice versa. By printing such materials, a gradient of stiffness or softness can be reached. Alternatively, by printing such materials graded stiffness from soft to medium or medium to soft as well as from stiff to medium or medium to stiff can be reached.

In case that no one or more additives are present, the first flowable composition consists of the first material and/or the second flowable composition consists of the second material.

Specifically, in case the first material and the second material are identical, the first flowable composition or the second flowable composition comprises one or more additives. In case the first flowable composition and the second flowable composition comprise one or more additives, the additives comprised in the first flowable composition and the second flowable composition have to be different as the first material and the second material are identical.

Alternatively, in case the first material and the second material are different from each other, the first flowable composition and/or the second flowable composition comprises one or more additives. The one or more additives are identical or different from each other.

Preferably, the one or more additives are comprised in the first flowable composition and/or in the second flowable composition in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v.

More preferably, the one or more additives are bioactive compounds such as a pharmaceutical active compounds/therapeutic agents/drugs. The bioactive compounds may be physical, chemical, or biological substances which can be used in the treatment, cure, prophylaxis, or prevention of a pathological condition, e.g. a disease or disorder, or which can be used to otherwise enhance physical, psychical or mental well-being.

Even more preferably, the one or more additives are selected from the group consisting of inorganic fillers, minerals, cells, viruses, spores, hyaluronic acid, dyes, proteins, lipids, drugs, activated carbon, cell culture materials.

Still even more preferably,
(i) the minerals are selected from the group consisting of apatite, clay, hydroxyapatite, graphene, carbon nanotubes, and silicate nanoparticles,
(ii) the cells are selected from the group consisting of bone cells, cartilage cells, neuronal cells, muscle cells, and stem cells,
(iii) the dye is selected from the group consisting of a synthetic dye, an inorganic dye, and an organic dye,
(iv) the protein is selected from the group consisting of an enzyme, an antibody, a hormone, and an antigen,
(v) the lipid is selected from the group consisting of a cholesterol, a steroid, a wax, and an oil,
(vi) the drug is selected from the group consisting of a growth-stimulating agent, an anti-inflammatory agent, an antimicrobial agent, and an antiviral agent, and/or
(vii) the activated carbon is selected from the group consisting of powdered activated carbon (PAC), granular activated carbon (GAC) and extruded activated carbon (EAC).

Most preferably,
(i) the apatite is selected from the group consisting of hydroxyapatite, fluorapatite, and chlorapatite,
(ii) the synthetic dye is an azo compound,
(iii) the inorganic dye is a metal salt, and/or
(iv) the organic dye is selected from the group consisting of a fluorescein dye and a rhodamine dye.

Examples of growth-stimulating agents/growth factors are cytokines or hormones that bind to specific receptors on the surface of their target cells. Specific examples of suitable growth-stimulating agents/growth factors include, but are not limited to, bone morphogenetic proteins (BMPs), epidermal growth factors (EGF), erythropoietin (EPO), fibroblast growth factor (FG), granulocyte-colony stimulating factor (G-CSF), granulocyte-macrophage stimulating factor (GM-CSF), growth differentiation factor-9 (GDF9), hepatocyte growth factor (HGF), hepatoma dervived growth factor (HDGF), insulin-like growth factor (HDGF), insulin-like growth factor (IGF), myostatin (GDF8), nerve-growth factor (NGF), platelet-dervived growth factor (PDGF), transforming growth factor alpha (TGF-α), transforming growth factor beta (TGF-β), and vascular ecdothelial growth factor (VEGF).

Further examples of useful dyes belong to, but are not limited to, the group of a acridine dyes such as acridine orange or acridine yellow, anthrachinone dyes such as Alizarin, Anthrapurpurin, Carminic acid, Disperse Red 11, Disperse Red 9, Indathrene blue RS, Morindone, Oil blue 35, Oil blue A, Quinizarine Green SS, Solven violet 13 or Vat Yellow 4, diarylmethane dyes such as the diarylmethane dye auramine 0 or triarylmethanes such as Aluminon, Aniline Blue WS, Aurin, Brilliant Blue FCF, Brilliant Green, Bromocresol green, Bromocresol purple, Bromophenol blue, Bromothymol blue, Bromosulphtalein, Chlorophenol red, Chromoxane cyanin R, Coomassie, Cresol red, Crystal violet lactone, Ethyl Green, Fast Green FCF, Fluoran, Fuchsin, Fuchsin acid, Green S, Light Green SF yellowish, Malachite green, Methyl violet, Methyl blue, Methylrosaniline, New fuchsine, pararosaniline, Patent Blue V, Phenol red, Phenolphtalein, Rose bengal, Thymolphtalein, Victoria blue BO, Xylene cyanol or Xylenol orange, azo dyes such as Alizarine Yellow R, Allura Red AC, Amaranth, Amido black 10 B, Aniline Yellow, Azo rubine, Biebrich scarlet, Bismarck brown Y, Black 7984, Brilliant black BN, Brown FK, Brown HT, Chrysoine resorcinol, Citrus red 2, Congo red, D&C Red 33, Disperse Orange 1, Eriochrome Black T, Fast Yellow AB, Hydroxynaphtol blue, Janus Green B, Lithol Rubine BK, Lithiol Rubine BK, Methyl orange, Methyl Red, Methyl yellow, Mordant Red 19, Oil Red O, Oil Yellow DE, Orange B, Orange G, Orange GGN, Para Red, Ponceau 2R, Ponceau 4R, Ponceau 6R, Ponceau S, Prontosil, Red 2G, Scarlet GN, Solvent Red 164, Solvent Red 26, Solvent Yellow 124, Sudan Black B, Sudan I, Sudan II, Sudan III, Sudan IV, Sudan Red 7B, Sudan Red G, Sudan Yellow 3G, Sudan Yellow FCF, Tartrazine, Tropaeolin OO, Tropaeolin OOO or Trypan blue, cyanin dyes (or phtalocyanines) such as Alcian blue, Luxol fast blue, Direct blue 86, Direct blue 199, Phtalocyanine blue BN or Phtalocyanine green GN, azin dyes such as Neutral Red or Safranin, Nitro dyes such as picric acid and martius yellow, indolphenol dyes such as dichlorophenolindophenol, oxazin dyes such as nile blue, nile red, gallocyanin, gallamin blue or celestin blue, thiazin dyes such as methylene blue or new methylene blue or toluidine blue O, xanthene dyes or derivatives thereof including fluorescein, eosins such as Eosin Y and Eosin B and rhodamines such as Rhodamine B, Rhodamine 6G, Rhodamine 123, pyronin dyes such as Pyronin B and Pyronin Y, tetramethylrhodamine (TAMRA) and its isothiocyanate derivative (TRITC), sulforhodamine 101 and its sulfonyl chloride form Texas Red and Rhodamine Red or newer fluorophores such as Alexa dyes, e.g. Alexa 546, Alexa 555, Alexa 633, or Dylight dyes, e.g. DyLight 549, DyLight 633, or a mixture thereof.

In at least one preferred embodiment,
the first material is comprised in the first flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and/or
the second material is comprised in the second flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

More preferably the first material is comprised in the first flowable composition in an amount of between 0.5 w/v and 20 w/v, e.g. 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 w/v, and/or the second material is comprised in the second flowable composition in an amount of between 0.5 w/v and 20 w/v, e.g. 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 w/v.

Even more preferably, the first material is comprised in the first flowable composition in an amount of between 3 w/v and 10 w/v, e.g. 3, 3.5, 4, 4.5, 5, 5.5, 6, 6.5, 7, 7.5, 8, 8.5, 9, 9.5, or 10 w/v, and/or the second material is comprised in the second flowable composition in an amount of between 3 w/v and 10 w/v, e.g. 3, 3.5, 4, 4.5, 5, 5.5, 6, 6.5, 7, 7.5, 8, 8.5, 9, 9.5, or 10 w/v.

In at least one preferred embodiment, the first material is selected from the group consisting of a silk polypeptide, hyaluronic acid, silicone, collagen, alginate, gelatin, gelatin with methacrylated groups (GelMA), heparin, chondroitin sulfate, chitosan, and cellulose such as nanocellulose, and/or the second material is selected from the group consisting of a silk polypeptide, hyaluronic acid, silicone, collagen, alginate, gelatin, gelatin with methacrylated groups (GelMA), heparin, chondroitin sulfate, chitosan, and cellulose such as nanocellulose.

More preferably, the first material comprised in the first flowable composition is a silk polypeptide or the second material comprised in the second flowable composition is a silk polypeptide. Even more preferably, the first material comprised in the first flowable composition is a silk polypeptide and the second material comprised in the second flowable composition is a silk polypeptide. Still even more preferably, the first material comprised in the first flowable composition is a silk polypeptide and the second material comprised in the second flowable composition is a silk polypeptide, wherein the first flowable composition and/or the second flowable composition comprises one or more additives. In case where the first material and the second material is a silk polypeptide and the first flowable composition and the second flowable composition comprises one or more additives, the additives differ from each other.

Thus, in at least one particular embodiment, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition.

Specifically, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition,
wherein
the one or more additives are comprised in the first flowable composition and/or in the second flowable composition in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v.

Specifically, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition,
wherein
the silk polypeptide is comprised in the first flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and/or
the silk polypeptide is comprised in the second flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

More specifically, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition,
wherein
the one or more additives are comprised in the first flowable composition and/or in the second flowable composition in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v, and
the silk polypeptide is comprised in the first flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and
the silk polypeptide is comprised in the second flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

Preferably, the one or more additives are selected from the group consisting of inorganic fillers, minerals, cells, viruses, spores, hyaluronic acid, dyes, proteins, lipids, drugs, activated carbon, cell culture materials, or combinations thereof More preferably, the one or more additives are apatite such as fluorapatite and cells. The fluorapatite is present in particle form.

In one specific embodiment, the additive, in particular the bioactive compound, is encapsulated in silk polypeptide capsules (as described in EP1757276). Specifically, the additive, in particular the bioactive compound, is comprised in the silk polypeptide capsules, e.g. in the inner lumen and/or matrix of the silk capsules, and/or attached to the silk polypeptide capsules, e.g. the shell of the silk capsules.

In one another specific embodiment, the additive, in particular the bioactive compound, is coated with silk polypeptides. In the examples, fluorapatite particles coated with the silk polypeptide C₁₆ in which all glutamic acid residues are replaced by lysine ones (κ16) were incorporated into a silk polypeptide C₁₆ hydrogel for printing (first flowable composition). Alternatively, cells as well as fluorapatite particles coated with the silk polypeptide C₁₆ in which all glutamic acid residues are replaced by lysine ones (κ16) were incorporated into a silk polypeptide C₁₆ hydrogel for printing (first flowable composition). Additionally, a silk polypeptide C₁₆ hydrogel was used (second flowable composition).

In at least one preferred embodiment,
the first flowable composition is a hydrogel, and/or
the second flowable composition is a hydrogel.

Preferably, the hydrogel is a silk polypeptide hydrogel.

Thus, in at least one particular embodiment, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel.

Specifically, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel,
wherein
the one or more additives are comprised in the first hydrogel and/or in the second hydrogel in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v.

Specifically, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel,
wherein
the silk polypeptide is comprised in the first hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and/or
the silk polypeptide is comprised in the second hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

More specifically, the reservoir comprises
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel,
wherein
the one or more additives are comprised in the first hydrogel and/or in the second hydrogel in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v, and
the silk polypeptide is comprised in the first hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and
the silk polypeptide is comprised in the second hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

Preferably, the one or more additives are selected from the group consisting of inorganic fillers, minerals, cells, viruses, spores, hyaluronic acid, dyes, proteins, lipids, drugs, activated carbon, cell culture materials, or combinations thereof.

More preferably, the one or more additives are apatite such as fluorapatite and cells. The fluorapatite is present in particle form.

In one specific embodiment, the additive, in particular the bioactive compound, is encapsulated in silk polypeptide capsules (as described in EP1757276). Specifically, the additive, in particular the bioactive compound, is comprised in the silk polypeptide capsules, e.g. in the inner lumen and/or matrix of the silk capsules, and/or attached to the silk polypeptide capsules, e.g. the shell of the silk capsules.

In one another specific embodiment, the additive, in particular the bioactive compound, is coated with silk polypeptides. In the examples, fluorapatite particles coated with the silk polypeptide C₁₆ in which all glutamic acid residues are replaced by lysine ones (κ16) were incorporated into a silk polypeptide C₁₆ hydrogel for printing (first flowable composition). Alternatively, cells as well as fluorapatite particles coated with the silk polypeptide C₁₆ in which all glutamic acid residues are replaced by lysine ones (κ16) were incorporated into a silk polypeptide C₁₆ hydrogel for printing (first flowable composition). Additionally, a silk polypeptide C₁₆ hydrogel was used (second flowable composition).

It is preferred that the silk polypeptide is a recombinant/synthetic silk polypeptide. The (recombinant/synthetic) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide, more particularly a recombinant spider silk polypeptide.

It is further (alternatively or additionally) more preferred that the silk polypeptide is a polypeptide with an amino acid sequence which comprises or consists of at least 50%, 60%, 65%, 70%, 75%, 80%, 85%, 90%, 95%, or 99% multiple copies of repetitive units or of even 100% multiple copies of repetitive units. Said repetitive units may be identical or different.

It is also (alternatively or additionally) more preferred that the silk polypeptide comprises at least two identical repetitive units. For example, the silk polypeptide comprises between 2 to 96 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 repetitive units.

It is also still (alternatively or additionally) more preferred that the silk polypeptide consists of between 40 to 3000 amino acids. It is even more preferred that the silk polypeptide consists of between 40 to 1500 amino acids or between 60 to 1200 amino acids. It is most preferred that the silk polypeptide consists of between 100 to 600 amino acids.

It is even more preferred that the repetitive units are independently selected from module C having an amino acid sequence according to SEQ ID NO: 1 or variants thereof, module C^{Cys} having an amino acid sequence according to SEQ ID NO: 2 or variants thereof, module C^{K} having an amino acid sequence according to SEQ ID NO: 3 or variants thereof, and module C^{Lys} having an amino acid sequence according to SEQ ID NO: 4 or variants thereof.

Module C^{Cys} (SEQ ID NO: 2) is a variant of module C (SEQ ID NO: 1). In this module, the amino acid Ser at position 25 has been replaced by the amino acid Cys. Module C^{K} (SEQ ID NO: 3) is also a variant of module C (SEQ ID NO: 1). Module C^{Lys} (SEQ ID NO: 4) is also a variant of module C (SEQ ID NO: 1). In this module, the amino acid Glu at position 20 has been replaced by the amino acid Lys. In one example, the silk polypeptide comprises 16 C^{K} modules (SEQ ID NO: 3). In one another example, the silk polypeptide comprises 16 modules, wherein the first module (N-terminal) or the last module (C-terminal) is a C^{Lys} module (SEQ ID NO: 4) and the 15 other modules are C modules (SEQ ID NO: 1).

The module C variant differs from the reference module C from which it is derived by up to 1, 2, 3, 4, 5, 6, 7, 8, 9, or 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterized by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C.

Preferably, the sequence identity is over a continuous stretch of at least 5, 10, 15, 18, 20, 24, 27, 28, 30, 34, or more amino acids, preferably over the whole length of the respective reference module C.

The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.

A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.

Additionally, the module C variant or fragment is only regarded as a module C variant or fragment within the context of the present invention, if the modifications with respect to the amino acid sequence on which the variant or fragment is based do not negatively affect the ability of the silk polypeptide of being printed (specifically in form of a gradient). The skilled person can readily assess whether the silk polypeptide comprising a module C variant or fragment is still capable of being printed (specifically in form of a gradient). In this respect, it is referred to the examples comprised in the experimental part of the present patent application.

C^{Cys}, C^{K}, or C^{Lys} variants may also be encompassed by the present invention. Regarding the C^{Cys}, C^{K}, or C^{Lys} variants, the same explanations/definitions apply which have been made with respect to the module C variant (see above).

It is still even more preferred that the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.

It is most preferred that the silk polypeptide is selected from the group consisting of C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys}, (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys}, (C)₈C^{Lys} (C)₁₆C^{Lys}, (C)₃₂C^{Lys}, (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.

Exemplarily silk polypeptides are the following: The silk polypeptide C₈ (8 times module C) has the amino acid sequence according to SEQ ID NO: 5. The silk polypeptide C₁₆ (16 times module C) has the amino acid sequence according to SEQ ID NO: 6. The silk polypeptide C₃₂ (32 times module C) has the amino acid sequence according to SEQ ID NO: 7. The silk polypeptide C₄₈ (48 times module C) has the amino acid sequence according to SEQ ID NO: 8.

Particularly, the above-described silk polypeptide consists exclusively of repetitive units. In other words, the silk polypeptide particularly does not comprise/is free of non-repetitive units.

The only component that can additionally be present as part of the silk polypeptide is a tag or moiety, e.g. allowing easy transcription of said silk polypeptide in expression systems and/or allowing easy isolation of said silk polypeptide from the expression systems. Said tag may be a his tag or a flag tag.

The content may further comprise additional flowable compositions, e.g. a third flowable composition comprising a third material, a fourth flowable composition comprising a fourth material, a fifth flowable composition comprising a fifth material, etc. These compositions may optionally comprise one or more additives.

In all of the above described embodiments, it is preferred that the reservoir is a cartridge, preferably a printer cartridge.

In a second aspect, the present invention relates to a printer comprising the cartridge according to the first aspect or a receptacle for such a cartridge. The printer may be a pressurization system to pressurize the content of the cartridge. Preferably, the printer is a 3D printer or an inkjet printer.

In the examples, a conical piston and a 14, 16, or 20 G needle were used. In addition, cartridges with an inner diameter of 14, 16, or 20 G were used. The printing can be carried out with a printing speed of 20 mm/s and 0.1 bar.

In a third aspect, the present invention relates to a method of dispensing a content from a reservoir, e.g. a reservoir or a cartridge according to the first aspect, comprising the step of: applying pressure to a content of the reservoir, so that the content is dispensed from the reservoir via an outlet, wherein the content comprises a first flowable composition comprising a first material, and a second flowable composition comprising a second material.

In at least one embodiment, during dispensing content from the reservoir, at least a part of the dispensed content comprises at the outlet the first flowable composition and the second flowable composition which leave the reservoir simultaneously through the outlet.

In at least one embodiment, the first flowable composition and the second flowable composition which leave the reservoir simultaneously through the outlet are deposited simultaneously on a target surface. The target surface may be a (polystyrene) dish, e.g. petri dish, or a plane surface.

In at least one particular embodiment, pressurisation of the content results in a non-plane boundary surface between the first flowable composition and second flowable composition.

In at least one specific embodiment, the boundary surface has a U-shaped cross section.

In at least one another specific embodiment, the content is dispensed from the reservoir through the outlet with a laminar flow. Especially, the boundary surface has a U-shaped cross section and the content is dispensed from the reservoir through the outlet with a laminar flow.

Preferably, a pressure of between 5000 and 20000 Pa, preferably of between 7000 and 15000 Pa, e.g. 10000 PA (=0.1 bar), is applied. More preferably, the content is dispensed from the reservoir with a (extrusion) velocity of between 2 mm/s and 100 mm/s, even more preferably of between 5 mm/s and 50 mm/s, and still even more preferably of between 5 mm/s and 20 mm/s, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mm/s. Alternatively, the content is dispensed from the reservoir with a specific flow rate.

In the examples, a conical piston and a 14, 16, or 20 G needle was used. In addition, cartridges with an inner diameter of 14, 16, or 20 G were used. The printing was carried out with a printing speed of 20 mm/s and 0.1 bar.

For simultaneous printing of the first flowable composition and the second flowable composition, it is preferred that the first and second materials comprised therein have flow points in the same order of magnitude.

In at least one preferred embodiment, the first flowable composition and/or the second flowable composition comprise one or more additives. Thus, the first flowable composition comprises, in addition to a first material, one or more additives and/or the second flowable composition comprises, in addition to a second material, one or more additives.

As mentioned above, the first flowable composition and the second flowable composition differ from each other. Thus, the first flowable composition and the second flowable composition may differ from each other in terms of the first and second material or in terms of the one or more additives comprised therein.

In one example, the first and second material is identical. In this case, the difference between the first flowable composition and second flowable composition lies within the one or more additives comprised therein.

In one another example, the first and second material is different. In this case, the difference between the first flowable composition and/or second flowable composition lies within the first and second material comprised therein. Optionally, one or more additives are comprised in the first flowable composition and/or second flowable composition. In case that one or more additives are part of the first flowable composition and/or second flowable composition, the one or more additives can be identical or different.

The difference between the first material and the second material may lie in the type, structure, concentration, and/or viscosity of the first material and the second material.

For example, the first material and the second material may differ in their mechanical properties. Specifically, the first material may be a stiff material and the second material may be a soft material, or vice versa. Alternatively, the first material may be a material between soft and stiff (i.e. medium) and the second material may be a soft material, or vice versa. Alternatively, the first material may be a material between soft and stiff (i.e. medium) and the second material may be a stiff material, or vice versa. By printing such materials, a gradient of stiffness or softness can be reached. Alternatively, by printing such materials graded stiffness from soft to medium or medium to soft as well as from stiff to medium or medium to stiff can be reached.

In case that no one or more additives are present, the first flowable composition consists of the first material and/or the second flowable composition consists of the second material.

Specifically, in case the first material and the second material are identical, the first flowable composition or the second flowable composition comprises one or more additives. In case the first flowable composition and the second flowable composition comprise one or more additives, the additives comprised in the first flowable composition and the second flowable composition have to be different as the first material and the second material are identical.

Alternatively, in case the first material and the second material are different from each other, the first flowable composition and/or the second flowable composition comprises one or more additives. The one or more additives are identical or different from each other.

Preferably, the one or more additives are comprised in the first flowable composition and/or in the second flowable composition in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v.

More preferably, the one or more additives are bioactive compounds such as a pharmaceutical active compounds/therapeutic agents/drugs. The bioactive compounds may be physical, chemical, or biological substances which can be used in the treatment, cure, prophylaxis, or prevention of a pathological condition, e.g. a disease or disorder, or which can be used to otherwise enhance physical, psychical or mental well-being.

Even more preferably, the one or more additives are selected from the group consisting of inorganic fillers, minerals, cells, viruses, spores, hyaluronic acid, dyes, proteins, lipids, drugs, activated carbon, cell culture materials.

Still even more preferably,
(i) the minerals are selected from the group consisting of apatite, clay, hydroxyapatite, graphene, carbon nanotubes, and silicate nanoparticles,
(ii) the cells are selected from the group consisting of bone cells, cartilage cells, neuronal cells, muscle cells, and stem cells,
(iii) the dye is selected from the group consisting of a synthetic dye, an inorganic dye, and an organic dye,
(iv) the protein is selected from the group consisting of an enzyme, an antibody, a hormone, and an antigen,
(v) the lipid is selected from the group consisting of a cholesterol, a steroid, a wax, and an oil,
(vi) the drug is selected from the group consisting of a growth-stimulating agent, an anti-inflammatory agent, an antimicrobial agent, and an antiviral agent, and/or
(vii) the activated carbon is selected from the group consisting of powdered activated carbon (PAC), granular activated carbon (GAC) and extruded activated carbon (EAC).

Most preferably,
(i) the apatite is selected from the group consisting of hydroxyapatite, fluorapatite, and chlorapatite,
(ii) the synthetic dye is an azo compound,
(iii) the inorganic dye is a metal salt, and/or
(iv) the organic dye is selected from the group consisting of a fluorescein dye, and a rhodamine dye.

In at least one preferred embodiment,
the first material is comprised in the first flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and/or
the second material is comprised in the second flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

More preferably the first material is comprised in the first flowable composition in an amount of between 0.5 w/v and 20 w/v, e.g. 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 w/v, and/or the second material is comprised in the second flowable composition in an amount of between 0.5 w/v and 20 w/v, e.g. 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 w/v.

Even more preferably, the first material is comprised in the first flowable composition in an amount of between 3 w/v and 10 w/v, e.g. 3, 3.5, 4, 4.5, 5, 5.5, 6, 6.5, 7, 7.5, 8, 8.5, 9, 9.5, or 10 w/v, and/or the second material is comprised in the second flowable composition in an amount of between 3 w/v and 10 w/v, e.g. 3, 3.5, 4, 4.5, 5, 5.5, 6, 6.5, 7, 7.5, 8, 8.5, 9, 9.5, or 10 w/v.

In at least one preferred embodiment,
the first material is selected from the group consisting of a silk polypeptide, hyaluronic acid, silicone, collagen, alginate, gelatin, gelatin with methacrylated groups (GelMA), heparin, chondroitin sulfate, chitosan, and cellulose such as nanocellulose, and/or the second material is selected from the group consisting of a silk polypeptide, hyaluronic acid, silicone, collagen, alginate, gelatin, gelatin with methacrylated groups (GelMA), heparin, chondroitin sulfate, chitosan, and cellulose such as nanocellulose.

More preferably, the first material comprised in the first flowable composition is a silk polypeptide or the second material comprised in the second flowable composition is a silk polypeptide. Even more preferably, the first material comprised in the first flowable composition is a silk polypeptide and the second material comprised in the second flowable composition is a silk polypeptide. Still even more preferably, the first material comprised in the first flowable composition is a silk polypeptide and the second material comprised in the second flowable composition is a silk polypeptide, wherein the first flowable composition and/or the second flowable composition comprises one or more additives. In case where the first material and the second material is a silk polypeptide and the first flowable composition and the second flowable composition comprises one or more additives, the additives differ from each other.

Thus, in at least one particular embodiment, the method of dispensing a content from a reservoir comprises the step of:
applying pressure to a content of the reservoir, so that the content is dispensed from the reservoir via an outlet, wherein the content comprises
(a) a first flowable composition comprising a silk polypeptide and one or more additives, and a second flowable composition comprising a silk polypeptide, or
(b) a first flowable composition comprising a silk polypeptide, and a second flowable composition comprising a silk polypeptide and one or more additives, or
(c) a first flowable composition comprising a silk polypeptide and one or more additives, and a second flowable composition comprising a silk polypeptide and one or more additives,
wherein the one or more additives comprised in the first flowable composition differ from
the one or more additives comprised in the second flowable composition.

Specifically, the method of dispensing a content from a reservoir comprises the step of: applying pressure to a content of the reservoir, so that the content is dispensed from the reservoir via an outlet, wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition,
wherein
the one or more additives are comprised in the first flowable composition and/or in the second flowable composition in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v.

Specifically, the method of dispensing a content from a reservoir comprises the step of:
applying pressure to a content of the reservoir, so that the content is dispensed from the reservoir via an outlet, wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition,
wherein
the silk polypeptide is comprised in the first flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and/or
the silk polypeptide is comprised in the second flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

More specifically, the method of dispensing a content from a reservoir comprises the step of:
applying pressure to a content of the reservoir, so that the content is dispensed from the reservoir via an outlet, wherein the content comprises
(a) a first flowable composition comprising a silk polypeptide and one or more additives, and
   a second flowable composition comprising a silk polypeptide, or
(b) a first flowable composition comprising a silk polypeptide, and
   a second flowable composition comprising a silk polypeptide and one or more additives, or
(c) a first flowable composition comprising a silk polypeptide and one or more additives, and
   a second flowable composition comprising a silk polypeptide and one or more additives, wherein the one or more additives comprised in the first flowable composition differ from the one or more additives comprised in the second flowable composition,
wherein
the one or more additives are comprised in the first flowable composition and/or in the second flowable composition in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v, and
the silk polypeptide is comprised in the first flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and
the silk polypeptide is comprised in the second flowable composition in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

Preferably, the one or more additives are selected from the group consisting of inorganic fillers, minerals, cells, viruses, spores, hyaluronic acid, dyes, proteins, lipids, drugs, activated carbon, cell culture materials, or combinations thereof.

More preferably, the one or more additives are apatite such as fluorapatite and cells. The fluorapatite is present in particle form.

In one specific embodiment, the additive, in particular the bioactive compound, is encapsulated in silk polypeptide capsules (as described in EP1757276). Specifically, the additive, in particular the bioactive compound, is comprised in the silk polypeptide capsules, e.g. in the inner lumen and/or matrix of the silk capsules, and/or attached to the silk polypeptide capsules, e.g. the shell of the silk capsules.

In one another specific embodiment, the additive, in particular the bioactive compound, is coated with silk polypeptides. In the examples, fluorapatite particles coated with the silk polypeptide C₁₆ in which all glutamic acid residues are replaced by lysine ones (κ16) were incorporated into a silk polypeptide C₁₆ hydrogel for printing (first flowable composition). Alternatively, cells as well as fluorapatite particles coated with the silk polypeptide C₁₆ in which all glutamic acid residues are replaced by lysine ones (κ16) were incorporated into a silk polypeptide C₁₆ hydrogel for printing (first flowable composition). Additionally, a silk polypeptide C₁₆ hydrogel was used (second flowable composition).

In at least one preferred embodiment,
the first flowable composition is a hydrogel, and/or
the second flowable composition is a hydrogel.

Preferably, the hydrogel is a silk polypeptide hydrogel.

Thus, in at least one particular embodiment, the method of dispensing a content from a reservoir comprises the step of:
applying pressure to a content of the reservoir, so that the content is dispensed from the reservoir via an outlet, wherein the content comprises
(a) a first hydrogel comprising a silk polypeptide and one or more additives, and a second hydrogel comprising a silk polypeptide, or
(b) a first hydrogel comprising a silk polypeptide, and a second hydrogel comprising a silk polypeptide and one or more additives, or
(c) a first hydrogel comprising a silk polypeptide and one or more additives, and a second hydrogel comprising a silk polypeptide and one or more additives, wherein the one or more additives comprised in the first hydrogel differ from the one or more additives comprised in the second hydrogel.

Specifically, the method of dispensing a content from a reservoir comprises the step of: applying pressure to a content of the reservoir, so that the content is dispensed from the reservoir via an outlet, wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel,
wherein
the one or more additives are comprised in the first hydrogel and/or in the second hydrogel in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v.

Specifically, the method of dispensing a content from a reservoir comprises the step of: applying pressure to a content of the reservoir, so that the content is dispensed from the reservoir via an outlet, wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel,
wherein
the silk polypeptide is comprised in the first hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and/or
the silk polypeptide is comprised in the second hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

More specifically, the method of dispensing a content from a reservoir comprises the step of applying pressure to a content of the reservoir, so that the content is dispensed from the reservoir via an outlet, wherein the content comprises
(a) a first hydrogel comprising a silk polypeptide and one or more additives, and
   a second hydrogel comprising a silk polypeptide, or
(b) a first hydrogel comprising a silk polypeptide, and
   a second hydrogel comprising a silk polypeptide and one or more additives, or
(c) a first hydrogel comprising a silk polypeptide and one or more additives, and
   a second hydrogel comprising a silk polypeptide and one or more additives, wherein the one or more additives comprised in the first hydrogel differ from the one or more additives comprised in the second hydrogel,
wherein
the one or more additives are comprised in the first hydrogel and/or in the second hydrogel in an amount of between 0.01 w/v and 10 w/v, more preferably between 0.5 w/v and 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v, and
the silk polypeptide is comprised in the first hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, and
the silk polypeptide is comprised in the second hydrogel in an amount of between 0.01 w/v and 30 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v.

Preferably, the one or more additives are selected from the group consisting of inorganic fillers, minerals, cells, viruses, spores, hyaluronic acid, dyes, proteins, lipids, drugs, activated carbon, cell culture materials, or combinations thereof.

More preferably, the one or more additives are apatite such as fluorapatite and cells. The fluorapatite is present in particle form.

In one specific embodiment, the additive, in particular the bioactive compound, is encapsulated in silk polypeptide capsules (as described in EP1757276). Specifically, the additive, in particular the bioactive compound, is comprised in the silk polypeptide capsules, e.g. in the inner lumen and/or matrix of the silk capsules, and/or attached to the silk polypeptide capsules, e.g. the shell of the silk capsules.

In one another specific embodiment, the additive, in particular the bioactive compound, is coated with silk polypeptides. In the examples, fluorapatite particles coated with the silk polypeptide C₁₆ in which all glutamic acid residues are replaced by lysine ones (κ16) were incorporated into a silk polypeptide C₁₆ hydrogel for printing.

The content may further comprise additional flowable compositions, e.g. a third flowable composition comprising a third material, a fourth flowable composition comprising a fourth material, a fifth flowable composition comprising a fifth material, etc. These compositions may optionally comprise one or more additives.

As to the preferred embodiments of the silk polypeptide, it is referred to the first aspect of the present invention.

As mentioned above, the reservoir is in a preferred embodiment a syringe. In this embodiment, the content comprised therein can be removed from/squeezed out the syringe by hand/manually, e.g. by driving the piston comprised therein relative to the syringe body. The syringe is suitable for application of a dermal filler. Thus, in a preferred embodiment, the syringe comprises a dermal filler which can be applied. In a more preferred embodiment, the syringe comprises a first dermal filler as first flowable composition and a second dermal filler as second flowable composition, wherein the first and second flowable composition differ from each other.

In a fourth aspect, the present invention relates to a kit comprising the reservoir or the cartridge according to the first aspect or the printer according to the second aspect. Preferably, the kit further comprises a needle, a hose, and/or a closing plug, preferably a conical or round plug.

In a fifth aspect, the present invention relates to the use of the reservoir, preferably cartridge, according to the first aspect or the printer according to the second aspect for bioprinting.

Bioprinting is a manufacturing process that combines different materials, such as biomaterials, optionally biomaterials and one or more additives, such as cells and/or growth factors, with each other. These biomaterials can be used to create tissue-like structures that mimic natural tissue. Similar to inkjet printing, the bioprinter prints layer by layer. As with tissue engineering, biological and biologically functional tissues are produced in the laboratory. A broad field of potential applications in medicine and bioengineering is opening up for this novel technology. The technology has already seen recent progress in the production of cartilage tissue for reconstruction and regeneration.

In a sixth aspect, the present invention relates to the use of the reservoir, preferably cartridge, according to the first aspect or the printer according to the second aspect for biofabrication.

Biofabrication refers to the development and cultivation of bioartificial, cellular tissues using a variety of biotechnological, biomedical, and materials science and engineering methods, processes, and procedures. The present inventors used bioprinting, in particular 3D bioprinting, to fabricate a bioproduct, e.g. fluorapatite loaded silk polypeptide hydrogels or fluorapatite and cell loaded silk polypeptide hydrogels.

In this respect, it should be noted that 3D bioprinting is an additive manufacturing process for the production of living tissue. For example, hydrogels loaded with living cells are printed layer-by-layer to generate a precursor structure that can mature into functional, biological tissue in subsequent culturing steps. Compared to other biofabrication methods, such as cell colonization or molding, 3D bioprinting has several advantages that are important for the production of complex tissues. These include the spatially controlled and reproducible placement of different biomaterials and cells in a biological structure, as well as the production of patient-specific 3D geometries. Another advantage is the high degree of automation and digitization of the process. Specifically, dispense plotted gradients were obtained by the present inventors using recombinant silk hydrogels loaded with fluorapatite or using recombinant silk hydrogels loaded with fluorapatite as well as BALB/3T3 fibroblasts (see experimental section). With this experiment, the present inventors confirmed the possibility to generate particle loaded and cell loaded gradient hydrogels with controllable properties out of one printing cartridge, which is useful for biofabrication of soft-hard tissue interfaces.

Preferably, the biofabrication includes tissue, bone, tendon, or cartilage construction.

In a seventh aspect, the present invention relates to the use of the reservoir, preferably cartridge, according to the first aspect or the printer according to the second aspect for tissue engineering applications.

Tissue engineering is a biomedical engineering discipline that uses a combination of cells, engineering, materials methods, and suitable biochemical and physicochemical factors to restore, maintain, improve, or replace different types of biological tissues.

In particular, tissue engineering (TE) has emerged as a useful approach to treat tissue damages caused by diseases and trauma, which has shown many advantages as compared to conventional treatment strategies. To afford desirable therapeutic outcome, scaffolds prepared from various kinds of biomaterials have been used for TE to accommodate sufficient amount of cells and to control cell function. As a unique type of scaffolds, hydrogels have been frequently used for TE because of their similar 3D structures to the native extracellular matrix (ECM), as well as their tunable biochemical and biophysical properties to control cell functions such as cell adhesion, migration, proliferation, and differentiation.

In an eighth aspect, the present invention relates to the use of the reservoir, preferably cartridge, according to the first aspect or the printer according to the second aspect for gradient formation. Preferably, the gradient is formed by printing. Gradient formation refers to a technique of distributing a composition so that the concentration or level of different biomaterials and/or additives comprised therein continuously changes across an area. These concentrations or gradients are specifically important for recapitulating native gradients of signalling molecules as well as varying levels of matrix stiffness that occurs in tissues. For example, various planar and 3D structures exhibiting continual gradients of materials, of cell densities, of growth factor concentrations, of hydrogel stiffness, and of porosities in horizontal and/or vertical direction, are possible. The composable fabrication of multifunctional gradients strongly supports the potential of the unique bioprinting system in numerous biomedical applications. Preferably, the gradient is a mineralization gradient.

In a further aspect, the present invention relates to a printable composition comprising a silk polypeptide and apatite.

Preferably,
the silk polypeptide is comprised in an amount of 0.01 w/v to 30 w/v, more preferably 0.5 w/v to 20 w/v, and even more preferably 3 w/v to 10 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 w/v, in the composition, and/or
the apatite is comprised in an amount of 0.01 w/v to 10 w/v, more preferably 0.5 w/v to 1 w/v, e.g. 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 w/v, in the composition.

As to the preferred embodiments of the silk polypeptide, it is referred to the first aspect of the present invention. The apatite is preferably selected from the group consisting of hydroxyapatite, fluorapatite, and chlorapatite. Specifically, the printable composition comprises a silk polypeptide hydrogel with embedded apatite. In the examples, fluorapatite particles coated with the silk polypeptide C₁₆ in which all glutamic acid residues are replaced by lysine ones (κ16) were incorporated into a silk polypeptide C₁₆ hydrogel for printing. Alternatively, cells as well as fluorapatite particles coated with the silk polypeptide C₁₆ in which all glutamic acid residues are replaced by lysine ones (κ16) were incorporated into a silk polypeptide C₁₆ hydrogel for printing.

The present invention is summarized as follows:

1. A reservoir comprising
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
a first flowable composition comprising a first material, and
a second flowable composition comprising a second material.
   2. The reservoir of item 1, wherein the reservoir is configured such that during a dispensing operation for dispensing content from the reservoir, at least a part of the dispensed content comprises at the outlet the first flowable composition and the second flowable composition which leave the reservoir simultaneously through the outlet.
   3. The reservoir of item 2, wherein the first flowable composition and the second flowable composition which leave the reservoir simultaneously through the outlet can be deposited simultaneously on a target surface.
   4. The reservoir of any one of items 1 to 3, wherein the first flowable composition and the second flowable composition have a common boundary surface between them.
   5. The reservoir of item 4, wherein the common boundary surface is arranged within the interior space.
   6. The reservoir of items 4 or 5, wherein the reservoir is configured such that the common boundary surface comprises a plane central region when the content is not pressurised.
   7. The reservoir of any one of items 4 to 6, wherein the reservoir is configured such that the common boundary surface comprises a non-plane central region when the content is pressurised.
   8. The reservoir of any one of items 1 to 7, wherein the reservoir is configured such that pressurisation of the content results in a non-plane boundary surface between the first flowable composition and the second flowable composition.
   9. The reservoir of item 8, wherein the non-plane boundary surface comprises a concave central region as seen along a dispensing direction towards the outlet.
   10. The reservoir of items 8 or 9, wherein the non-plane boundary surface has a U-shaped cross section.
   11. The reservoir of any one of items 1 to 10, wherein the reservoir body reduces its inner cross section or tapers in a transition portion of the reservoir body as seen from the interior space towards the outlet along the flow direction from the interior space to the outlet.
   12. The reservoir of item 11, wherein the transition portion is curved.
   13. The reservoir of item 12, wherein the transition portion comprises an inner surface with a concavely curved region and a convexly curved region, as seen from within the reservoir body onto the inner surface.
   14. The reservoir of item 13, wherein the concavely curved portion is further away from the outlet or closer to the interior space than the convexly curved portion.
   15. The reservoir of items 13 or 14, wherein the inner cross section is greater in the concavely curved portion than in the convexly curved portion.
   16. The reservoir of any one of items 1 to 15, wherein an end portion of the reservoir or of the reservoir body is arranged between the outlet and the transition region.
   17. The reservoir of item 16, wherein an end of the end portion remote from the interior space defines the outlet.
   18. The reservoir of items 16 or 17, wherein an inner cross section of the end portion decreases, preferably continuously and/or monotonously, in the direction towards the outlet and/or away from the interior space.
   19. The reservoir of item 18, wherein the inner cross section of the end portion is conical.
   20. The reservoir of any one of items 1 to 19, wherein the interior space is continuous and/or has a constant cross-section.
   21. The reservoir of any one of items 1 to 20, wherein the reservoir is configured to dispense the content from the reservoir through the outlet with a laminar flow.
   22. The reservoir of any one of items 1 to 21, wherein the reservoir is configured to dispense the content from the interior space through the outlet such that the dispensed content can exhibit a gradient or change in the proportion of the first flowable composition and the proportion of the second flowable composition in the dispensed content.
   23. The reservoir of item 22, wherein the gradient changes along the dispensing direction or is constant.
   24. The reservoir of items 22 or 23, wherein the dispensed content comprises a region with a boundary between the first flowable composition and the second flowable composition.
   25. The reservoir of item 24, wherein the dispensed content comprises a first region consisting of the first flowable composition.
   26. The reservoir of items 24 or 25, wherein the dispensed content comprises a second region consisting of the second flowable composition.
   27. The reservoir of any one of items 24 to 26, wherein the dispensed content comprises a combined region comprising both, the first flowable composition and the second flowable composition.
   28. The reservoir of item 27, wherein
a proportion of the first flowable composition in the dispensed content increases within the combined region as seen from the combined region in a direction towards the first region and/or as seen from the combined region in a direction away from the second region along the dispensed content, and/or
a proportion of the second flowable composition in the dispensed content decreases within the same ratio as the first flowable composition increases within the combined region as seen from the combined region in a direction towards the first region and/or as seen from the combined region in a direction away from the second region along the dispensed content.
   29. The reservoir of items 27 or 28, wherein
a proportion of the second flowable composition in the dispensed content increases within the combined region as seen from the combined region in a direction towards the second region and/or as seen from the combined region in a direction away from the first region along the dispensed content, and/or
a proportion of the first flowable composition in the dispensed content decreases within the same ratio as the second flowable composition increases within the combined region as seen from the combined region in a direction towards the second region and/or as seen from the combined region in a direction away from the first region along the dispensed content.
   30. The reservoir of any one of items 24 to 29, wherein the boundary between the first flowable composition and the second flowable composition in the dispensed content is oblique relative to the dispensing direction.
   31. The reservoir of any one of items 1 to 30, wherein the interior space is circumferentially delimited by a cylindrical surface of the reservoir body.
   32. The reservoir of any one of items 1 to 31, wherein the reservoir body has an opening.
   33. The reservoir of item 32, wherein the opening is at an end of the reservoir body remote from the outlet.
   34. The reservoir of any one of items 1 to 33, wherein the interior space is delimited by a piston which is movably retained in the reservoir body.
   35. The reservoir of item 34, wherein the piston seals the interior space of the reservoir as seen from the opening.
   36. The reservoir of items 34 or 35, wherein the piston has a non-plane contact portion for contacting the content to pressurise the content.
   37. The reservoir of item 36, wherein the contact portion reduces its cross section or tapers as seen in a direction away from the opening.
   38. The reservoir of item 37, wherein the contact portion is conical.
   39. The reservoir of any one of items 34 to 38, wherein the piston is movable relative to the reservoir body, in particular towards the outlet, to pressurise the content.
   40. The reservoir of any one of items 34 to 39, wherein one of the first flowable composition and the second flowable composition is arranged between the piston and the other one of the first flowable composition and the second flowable composition.
   41. The reservoir of item 40, wherein the other one of the first flowable composition and the second flowable composition is arranged between the outlet and the one of the first flowable composition and the second flowable composition.
   42. The reservoir of any one of items 1 to 41, wherein the first flowable composition and/or the first material is a non-Newtonian fluid.
   43. The reservoir of any one of items 1 to 42, wherein the second flowable composition and/or the second material is a non-Newtonian fluid.
   44. The reservoir of any one of items 1 to 43, wherein the reservoir is a syringe.
   45. The reservoir of any one of items 1 to 44, wherein the reservoir comprises a luer connector.
   46. The reservoir of any one of items 1 to 45, wherein the outlet is configured to be releasably coupled to a needle or nozzle or wherein the outlet is formed by an end of a needle or nozzle.
   47. The reservoir of any one of items 1 to 46, wherein the reservoir is configured to be connected to a pneumatic circuit/motor, a hydraulic circuit/motor, or electric circuit/motor to pneumatically, hydraulically, or electronically drive the piston relative to the reservoir body.
   48. The reservoir of any one of items 1 to 47, wherein the reservoir comprises a pneumatic or hydraulic connector for operatively connecting the reservoir to a pneumatic or hydraulic pressurisation device.
   49. The reservoir of item 48, wherein the pneumatic or hydraulic pressurisation device is configured to pressurise the piston pneumatically or hydraulically, with the piston transferring the pressure to the content.
   50. The reservoir of any one of items 1 to 49, wherein the first flowable composition and/or the second flowable composition comprise one or more additives.
   51. The reservoir of any one of items 1 to 50, wherein the first material and the second material are identical or different from each other.
   52. The reservoir of item 51, wherein in case the first material and the second material are identical, the first flowable composition or the second flowable composition comprises one or more additives.
   53. The reservoir of item 51, wherein in case the first material and the second material are different from each other, the first flowable composition and/or the second flowable composition comprises one or more additives.
   54. The reservoir of any one of items 51 to 53, wherein at least one difference between the first material and the second material is in the type, structure, concentration, and/or viscosity of the first material and the second material.
   55. The reservoir of items 53 or 54, wherein in case one or more additives are comprised in the first flowable composition and in the second flowable composition, the one or more additives are identical or different from each other.
   56. The reservoir of any one of items 50 to 55, wherein the one or more additives are comprised in the first flowable composition in an amount of between 0.01 w/v and 10 w/v, preferably between 0.5 w/v and 1 w/v.
   57. The reservoir of any one of items 50 to 56, wherein the one or more additives are selected from the group consisting of inorganic fillers, minerals, cells, viruses, spores, hyaluronic acid, dyes, proteins, lipids, drugs, activated carbon, cell culture materials, or combinations thereof.
   58. The reservoir of item 57, wherein
(i) the minerals are selected from the group consisting of apatite, clay, hydroxyapatite, graphene, carbon nanotubes, and silicate nanoparticles,
(ii) the cells are selected from the group consisting of bone cells, cartilage cells, neuronal cells, muscle cells, and stem cells,
(iii) the dye is selected from the group consisting of a synthetic dye, an inorganic dye, and an organic dye,
(iv) the protein is selected from the group consisting of an enzyme, an antibody, a hormone, and an antigen,
(v) the lipid is selected from the group consisting of a cholesterol, a steroid, a wax, and an oil,
(vi) the drug is selected from the group consisting of a growth-stimulating agent, an anti-inflammatory agent, an antimicrobial agent, and an antiviral agent, and/or
(vii) the activated carbon is selected from the group consisting of powdered activated carbon (PAC), granular activated carbon (GAC) and extruded activated carbon (EAC).
   59. The reservoir of item 58, wherein
(i) the apatite is selected from the group consisting of hydroxyapatite, fluorapatite, and chlorapatite,
(ii) the synthetic dye is an azo compound,
(iii) the inorganic dye is a metal salt, and/or
(iv) the organic dye is selected from the group consisting of a fluorescein dye and a rhodamine dye.
   60. The reservoir of any one of items 1 to 59, wherein the first material is comprised in the first flowable composition in an amount of between 0.01 w/v and 30 w/v, preferably between 0.5 w/v and 20 w/v, more preferably between 3 w/v and 10 w/v.
   61. The reservoir of any one of items 1 to 60, wherein the second material is comprised in the second flowable composition in an amount of between 0.01 w/v and 30 w/v, preferably between 0.5 w/v and 20 w/v, more preferably between 3 w/v and 10 w/v.
   62. The reservoir of any one of items 1 to 61, wherein the first material is selected from the group consisting of a silk polypeptide, hyaluronic acid, silicone, collagen, alginate, gelatin, gelatin with methacrylated groups (GelMA), heparin, chondroitin sulfate, chitosan, and cellulose, preferably nanocellulose.
   63. The reservoir of any one of items 1 to 62, wherein the second material is selected from the group consisting of a silk polypeptide, hyaluronic acid, silicone, collagen, alginate, gelatin, gelatin with methacrylated groups (GelMA), heparin, chondroitin sulfate, chitosan, and cellulose, preferably nanocellulose.
   64. The reservoir of items 62 or 63, wherein the first material comprised in the first flowable composition is a silk polypeptide and the second material comprised in the second flowable composition is a silk polypeptide, wherein the first flowable composition or the second flowable composition comprises one or more additives.
   65. The reservoir of any one of items 62 to 64, wherein the silk polypeptide comprises at least two identical repetitive units.
   66. The reservoir of item 65, wherein the repetitive units are independently selected from the group consisting of module C having an amino acid sequence according to SEQ ID NO:
   1 or variants thereof, module C^{Cys} having an amino acid sequence according to SEQ ID NO: 2 or variants thereof, module C^{K} having an amino acid sequence according to SEQ ID NO: 3 or variants thereof, and module C^{LY}S having an amino acid sequence according to SEQ ID NO: 4 or variants thereof.
   67. The reservoir of item 66, wherein the silk polypeptide is selected from the group consisting of (C)ₘ, (C)ₘC^{Cys}, (C)ₘC^{K}, (C)ₘC^{Lys}, C^{Cys}(C)ₘ, C^{K}(C)ₘ, C^{Lys}(C)ₘ, (C^{Cys})ₘ, (C^{K})ₘ, and (C^{Lys})ₘ, wherein m is an integer of 2 to 96.
   68. The reservoir of item 67, wherein the silk polypeptide is selected from the group consisting of C₂, C₄, C₆, C₈, C₁₆, C₃₂, C₄₈, (C)₂C^{Cys}, (C)₄CC^{Y}s, (C)₆CC^{Y}s, (C)₈C^{Cys}(C)₁₆C^{Cys}, (C)₃₂C^{Cys}, (C)₄₈C^{Cys}, (C)₂C^{K}, (C)₄C^{K}, (C)₆C^{K}, (C)₈C^{K}, (C)₁₆C^{K}, (C)₃₂C^{K}, (C)₄₈C^{K}, (C)₂C^{Lys}, (C)₄C^{Lys}, (C)₆C^{Lys} (C)₈C^{Lys}, (C)₁₆C^{Lys}, (C)₃₂C^{Lys} (C)₄₈C^{Lys} C^{Cys}(C)₂, C^{Cys}(C)₄, C^{Cys}(C)₆, C^{Cys}(C)₈, C^{Cys}(C)₁₆, C^{Cys}(C)₃₂, C^{Cys}(C)₄₈, C^{K}(C)₂, C^{K}(C)₄, C^{K}(C)₆, C^{K}(C)₈, C^{K}(C)₁₆, C^{K}(C)₃₂, C^{K}(C)₄₈, C^{Lys}(C)₂, C^{Lys}(C)₄, C^{Lys}(C)₆, C^{Lys}(C)₈, C^{Lys}(C)₁₆, C^{Lys}(C)₃₂, C^{Lys}(C)₄₈, C^{Cys}₂, C^{Cys}₄, C^{Cys}₆, C^{Cys}₈, C^{Cys}₁₆, C^{Cys}₃₂, C^{Cys}₄₈, C^{K}₂, C^{K}₄, C^{K}₆, C^{K}₈, C^{K}₁₆, C^{K}₃₂, C^{K}₄₈, C^{Lys}₂, C^{Lys}₄, C^{Lys}₆, C^{Lys}₈, C^{Lys}₁₆, C^{Lys}₃₂, and C^{Lys}₄₈.
   69. The reservoir of any one of items 1 to 68, wherein the first flowable composition is a hydrogel.
   70. The reservoir of any one of items 1 to 69, wherein the second flowable composition is a hydrogel.
   71. The reservoir of any one of items 1 to 70, wherein the reservoir is a cartridge, preferably a printer cartridge.
   72. A printer comprising the cartridge of item 71 or a receptacle for such a cartridge.
   73. The printer of item 72, wherein the printer is a 3D printer or an inkjet printer.
   74. A method of dispensing a content from a reservoir, e.g. a reservoir of any one of items 1 to 70 or a cartridge of item 71, comprising the step of
applying pressure to a content of the reservoir, so that the content is dispensed from the reservoir via an outlet, wherein the content comprises a first flowable composition comprising a first material, and
a second flowable composition comprising a second material.
   75. The method of item 74, wherein during dispensing content from the reservoir, at least a part of the dispensed content comprises at the outlet the first flowable composition and the second flowable composition which leave the reservoir simultaneously through the outlet.
   76. The method of item 75, wherein the first flowable composition and the second flowable composition which leave the reservoir simultaneously through the outlet are deposited simultaneously on a target surface.
   77. The method of any one of items 74 to 76, wherein pressurisation of the content results in a non-plane boundary surface between the first flowable composition and second flowable composition.
   78. The method of item 77, wherein the boundary surface has a U-shaped cross section.
   79. The method of any one of items 74 to 78, wherein the content is dispensed from the reservoir through the outlet with a laminar flow.
   80. The method of any one of items 74 to 79, wherein a pressure between 50000 and 20000 Pa, preferably between 7000 and 15000 Pa, is applied.
   81. The method of any one of items 74 to 80, wherein the content is dispensed from the reservoir with a (extrusion) velocity of between 2 mm/s and 100 mm/s, preferably between 5 mm/s and 50 mm/s, more preferably 5 mm/s and 20 mm/s.
   82. The method of any one of items 74 to 81, wherein the content is dispensed from the reservoir with a specific flow rate.
   83. A kit comprising the reservoir of any one of items 1 to 70, the cartridge of item 71, or the printer of items 72 or 73.
   84. The kit of item 83, wherein the kit further comprises a needle, a hose, and/or a closing plug, preferably a conical or round plug.
   85. A printable composition comprising a silk polypeptide and apatite.
   86. The printable composition of item 85, wherein the silk polypeptide is comprised in an amount of 0.01 w/v to 30 w/v, preferably 0.5 w/v to 20 w/v, more preferably 3 w/v to 10 w/v and/or wherein the apatite is comprised in an amount of 0.01 w/v to 10 w/v, preferably 0.5 w/v to 1 w/v.
   87. The printable composition of items 85 or 86, wherein the printable composition comprises a silk polypeptide hydrogel with embedded apatite.
   88. The printable composition of any one of items 85 to 87, wherein the apatite is fluorapatite.
   89. Use of the reservoir of any one of items 1 to 70, the cartridge of item 71, or the printer of items 72 or 73 for bioprinting.
   90. The use of item 89 for 3D-bioprinting.
   91. Use of the reservoir of any one of items 1 to 70, the cartridge of item 71, or the printer of items 72 or 73 for biofabrication.
   92. The use of item 91, wherein the biofabrication includes tissue, bone, tendon, or cartilage construction.
   93. The use of item 92, wherein the tissue, bone, tendon, or cartilage construction includes the construction of tissue, bone, tendon, or cartridge replacement material.
   94. Use of the reservoir of any one of items 1 to 70, the cartridge of item 71, or the printer of items 72 or 73 for tissue engineering applications.
   95. Use of the reservoir of any one of items 1 to 70, the cartridge of item 71, or the printer of items 72 or 73 for gradient formation.
   96. The use of item 95, wherein the gradient is a mineralization gradient.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following figures are merely illustrative of the present invention and should not be construed to limit the scope of the invention as indicated by the appended claims in any way.

FIG. 1: Concentration of A and B blocks of an AB block system at the outlet a). Concentration profile of the cartridge during extrusion showing a backmixing effect, b) and a core-shell effect before the final material extrusion c).

FIG. 2: Photographs of 3D printed 3% w/v recombinant silk hydrogels: a) Gradient material from non-labelled (eADF4(C16)) to fluorescently labelled (eADF4(C16)/FITC-eADF4(C16)) in a Petri dish; b) Fluorescent image of the gradient material; c) Quantification of fluorescence output at distinct locations along the printed strand using three individual gradient constructs (circle, square triangle symbols are representing measurements for three individual scaffolds).

FIG. 3: Fluorapatite particles were blended with eADF4(C16) hydrogels. a) SEM image of 3% w/v hydrogels with 1% w/v FAp; b) SEM image of magnified FAp particles (one particle is indicated by an arrow); c) TEM image of eADF4(C16) fibrils, which build the hydrogel, intertwined FAp particles; d) Mean rheological amplitude sweep measurements of 3% w/v eADF4(C16) (black), 3% w/v eADF4(C16)+1% w/v FAp (dark grey) and 3% w/v eADF4(C16)+3% w/v FAp (light gray) hydrogels; e) Mean rheological amplitude sweep measurements of 3% w/v eADF4(C16) (black, upper curve), 3% w/v eADF4(C16)+1% w/v FAp (grey, middle curve) and 3% w/v eADF4(C16)+1% w/v kFAp (dark grey, lower curve) hydrogels; f) Mean rheological frequency sweep measurements of 3% w/v eADF4(C16) (black), 3% w/v eADF4(C16)+1% w/v FAp (grey) and 3% w/v eADF4(C16)+1% w/v kFAp (dark grey) hydrogels.

FIG. 4: Photograph of a 3D printed gradient of a recombinant silk hydrogels with and without fluorapatite particles: a) Gradient from 3% w/v eADF4(C16)+1% w/v kFAp (white) to 3% w/v eADF4(C16) (transparent); b) Light microscopy images at distinct locations of the construct showing decreasing particle concentration (higherst at position I and lowest at position VI), scale bar 50 μm.

FIG. 5: a) Quantified viability of BALB/3T3 fibroblasts using a DIN EN ISO 10993-5 and Cell Titer Blue assay. High density polyethylene served as positive and organotin-stabilized polyurethane as negative control for extract and direct contact test (n=3 for all conditions); b) Confocal laser scanning microscopy of BALB/3T3 fibroblasts along a printed gradient from 3% w/v eADF4(C16) to 3% w/v eADF4(C16)+1% w/v kFAp (position I lowest and position III highest concentration) using ethidium homodimer I (red)/calcein AM (green) staining. Not printed cell-loaded 3% w/v eADF4(C16)+1% kFAp w/v hydrogels served as control.

FIG. 6: Model set-up of a) block-system and mesh as slip wall and b) boundary conditions.

### EXAMPLES

The examples given below are for illustrative purposes only and do not limit the invention described above in any way.

### 1. MATERIALS & METHODS

### 1.1 Computational Flow Simulation

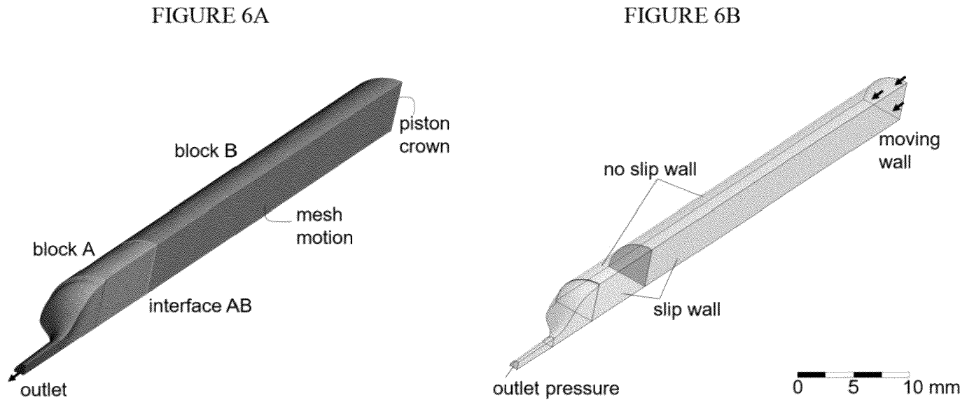

ANSYS Version15 CFX was used for the numerical simulation of gradient formation, which follows the Finite Volume Method as discretization technique (Malalasekera W, Versteeg HK. 2007 An Introduction to Computational Fluid Dynamics-The finite volume method. 2 ed. Essex, England: Pearson Education Limited). Due to symmetry, the printer cartridge was simplified to a quarter model as depicted in FIG. 6 under a). Only the fluid domain is modelled within the simulation set-up, where the inner wall of the real cartridge is mimicked by the outer contour of the fluid domain. The piston crown is approximated by a planar wall, as the distance to the outlet is large enough that the influence of the real piston geometry can be neglected. In addition, extrusion adapters, such as cannulas or cones were not taken into account to keep the model simple. The two phases (compositions) have been designed as two separate blocks, referred to as AB block system, with a flat contact interface in between. The ratio of the lengths of blocks A and B is 1:2. Both liquids A and B are modelled as homogenous non-Newtonian fluids. The power law according to Ostwald de Waele (Morrison FA. 2001 Understanding Rheology: Oxford University Press) is used for simulating the shear thinning properties of the recombinant silk hydrogel made of eADF4(C16): where τ[Pa] denotes the shear stress and γ·[s⁻¹] the shear rate. The flow consistency index is set to k=148.89 Pa*sⁿ with a flow behaviour index of n=0.2025. The material parameters are fitted from measured rheological data within a range of γ·=0.1 . . . 10 s−1.

The boundary conditions are defined as depicted in FIG. 6 under b). Slip boundary conditions are applied to the outer boundaries of the cartridge, whereas free slip behaviour is defined for the fluid interface of the domains and at symmetry planes of the quarter model.

During the printing process, the piston crown moves towards the outlet activating fluid flow motion. A displacement boundary condition is applied, causing the piston crown wall to move at a speed of ν=5 mm/s. Since domain B is compressed during the printing process, a mesh motion condition is defined for domain B. The transient flow simulation is performed using a second order backward Euler solution algorithm (ANSYS I. ANSYS CFX-Solver Theory Guide. Secondary. 16.2 ed. Canonsburg, USA: ANSYS, Inc.; 2015) with a time step size of Δt=0.1 s.

### 1.2 3D Dispense Plotting

A regenHU 3D Discovery Gen1 (Switzerland) bioplotter was used for dispense plotting, equipped with cartridges size 3 cc and according conical pistons. Luer lock conical needles were adapted to the cartridges with an inner diameter of 14 G, 16 G and 20 G. The different materials were filled in the cartridges manually using block volumes of 0.5-1 mL. Polystyrene Petri dishes were used as substrate (diameter 8 cm, Sarstedt, Germany). A round shaped monolayer construct was designed, and the according G-code generated using the regenHU BioCAD V1.1 printer software. The applied pressure was set to 0.1 bar (=10000 Pa) for all hydrogels. The printing speed was pre-set to 20 mm/s.

### 1.3 Recombinant Silk Protein Production, Protein Labelling and Hydrogel Preparation

The amino acid sequence of eADF4(C16) (SEQ ID NO: 6) is based on the consensus sequence of the repetitive core domain of the dragline silk fibroin 4 of the European garden spider Araneus diadematus, the so-called C-module sequence (GSSAAAAAAAASGPGGYGPENQGPSGPG GYGPGGP (SEQ ID NO: 1)). The C-module is repeated 16 times to yield eADF4(C16). In eADF4(κ16), all glutamic acid residues are replaced by lysine ones. Recombinant silk proteins were produced and purified as previously described (Huemmerich D, Helsen CW, Quedzuweit S, Oschmann J, Rudolph R, Scheibel T. 2004 Biochemistry 43 (42) 13604-12 and Doblhofer E, Scheibel T. 2015 Journal of Pharmaceutical Sciences 104 (3) 988-94). Hydrogels formed after protein dialysis and concentration adjustment by water removal using dialysis against PEG[19](Carl Roth, Germany). Covalent coupling of NHS-fluorescein (Thermo Scientific, Germany) to the amino-terminus of eADF4(C16) was conducted with a 10-fold molar excess of dye.

### 1.4 Fluorescence Analysis and Fluorescence Spectroscopy

Fluorescence images of printed gradient recombinant silk hydrogels with and without fluorescent labelling were recorded at CY2 mode (Ex 480/Em 530 nm, exposure 0.05) using an Ettan DIGE imager (GE Healthcare, Sweden).

In addition, fluorescence spectra (n=3 per sample) were recorded using a fluorescence spectrometer FP-6300 (JASCO, Germany) using an excitation wavelength of 488 nm at 20° C. 3 μL of hydrogel samples were taken at equal distances (3 cm) from the printed strands and resuspended in 0.25 mL 10 mM Tris buffer, pH 7.5. Maximum fluorescence values at 515 nm were plotted against the respective distance.

### 1.5 Fluorapatite Particle Synthesis

14.6 g CaCl₂) (Carl Roth, Germany), 14.3 g Na2IPO4 (Carl Roth, Germany) and 0.8 g NaF (Carl Roth, Germany) were mixed in dry state, and 40 mL of MilliQ water were added shortly before ultrasonication for 5 min at an energy intake of 18 kJ using a Sonoplus Ultrasonic Homogenizer (Bandelin, Germany) and a KE76 probe. Particles were washed with MilliQ water and air dried at 50° C. over night.

FAp particles were coated in aqueous solution using eADF4(κ16) dissolved in 6 M guanidine thiocyanate and dialysed against 10 mM Tris buffer, pH 7.5 for 16 h. The coating parameter were 10 mg particles in 1 mL protein solutions with 1 mg/ml for 4 h, followed by centrifugation at 13.000 rpm for 10 min and washing in MilliQ water.

### 1.6 Attenuated Total Reflectance-Fourier Transformation Infrared Spectroscopy

Attenuated Total Reflectance-Fourier Transformation Infrared (ATR-FTIR) spectra (n=3) were recorded in dry state, using a germanium crystal mounted on a Bruker Tensor 27 (Ettlingen, Germany) at a resolution of 2 cm-1 using 100 scans. An atmospheric compensation algorithm was used in OPUS 8.0 software to correct water vapour and carbon dioxide fluctuations during the measurement.

### 1.7 Microscopy

Fluorapatite particles were studied after carbon sputtering (20 nm) using SEM-imaging and a ZEISS Sigma 300 VP and Sigma 500 chamber equipped with an EDS detector (EDAX Pegasus and Octane Super Detector, 60 mm2 chip, Zeiss, Germany) at an acceleration voltage of 7.5 kV to excite the Kα shell. For SEM imaging of particle-loaded hydrogels, samples were freeze-dried to maintain pore structures before platinum sputtering (2 nm). Images were recorded using a Thermo Scientific (FEI) Apreo VS with a Field Emission Gun at 2 kV and a SE2-detector.

For transmission electron microscopy (TEM) images of fluorapatite (FAp) particles and hydrogels with FAp particles, samples were immobilized on Pioloform-coated 100-mesh copper grids (Plano GmbH, Germany) and stained with Uranyl acetate. JEM-2100 TEM (JEOL, Japan) was operated at 80 kV, and images were taken using a 4 000×4 000 charge-coupled device camera (UltraScan 4000, Gatan, USA) and Gatan Digital Micrograph software (version 1.83.842). Particle size was determined from 10 individual particles using ImageJ software (NHI, USA).

Light microscopy images were recorded in wet state using a Leica DM IL LED microscope (Leica, Germany) and processed using LAS 4.8 software (Leica, Germany).

Confocal laser scanning microscopy was carried out using a Leica CLSM TCS SP8 (Leica, Germany) and LAS software, and images were processed using ImageJ (NHI, USA).

### 1.8 Rheology

Rheological data were recorded using a Discovery Hybrid Rheometer 3 (TA, USA) using a plate-plate geometry (diameter 25 mm) with a sample volume of 500 μL (n=3) and a gap size of 500 μm at 25° C. A wet sponge adapter around the geometry served to prevent the premature drying of the hydrogels. Frequency sweep experiments were recorded at angular frequencies between 0.1-100 rad/s and 100-0.1 rad/s for recovery at 50% strain. Amplitude sweeps were recorded at 31.4 rad/s and a strain from 0.1-1000%.

### 1.9 Dynamic Light Scattering

Dynamic light scattering (DLS) was measured using a Litesizer™ 500 (Anton Paar, Austria). Diluted particle samples were recorded in 10 mM Tris buffer, pH 7.5 in omega cuvettes at 25° C. Mean values of zeta potentials were automatically calculated by the Kalliope software from internal spectra using the Smoluchowski model (Smoluchowski Mv. 1917 Z Phys Chem 92 129-68).

### 1.10 Cell Culture

Cytotoxicity was analyzed according to DIN EN ISO 10993-5 using BALB/3T3 mouse fibroblasts. BALB/3T3 mouse fibroblasts (ATCC, ACC210) were subsequently cultured in Dulbecco's Modified Eagle Media (Biochrom, Germany) with supplemented 10% v/v fetal calve serum (Bio&Sell, Germany), 1% v/v GlutaMax (Invitrogen, USA) and 0.1% v/v gentamycin sulphate (Sigma-Aldrich, Germany) at 37° C., 5% C02 and 95% relative humidity in a cell culture incubator (Thermo Scientific, Germany, HERAcell 150i). For the test, sub-confluent cultures were seeded at 25 000 cells/cm2 on treated tissue culture plates (TCP, Thermo Fisher Scientific, Germany, Nunclon) 24 h prior to the test (passage 9). Particles, high density polyethylene and organotin-stabilized polyurethane were UV treated for 30 min prior to use in cell culture (n=3). For extraction, 10 mg of fluorapatite and eADF4(k16)-coated fluorapatite particles were incubated in 1 mL cell culture media for 24 h at 37° C. For direct tests, between 10-80% of the test area were covered with the test species. To quantify cell viability, 10% v/v CellTiter-Blue reagent (Promega, Germany) were incubated on washed cells for 2.5 h. 100 μL of the sample were used to analyse the metabolism of resazurin to resofurin at 590 nm using a plate reader (Mithras LB 940, BertholdTechnologies, Germany). Significance was calculated using ANOVA statistic in Origin (Northampton, Massachusetts, USA) in a Tukey test with p<0.05. For gradient printing of cells, 106 cells/mL were seeded (at passage 10) with 15% cell culture media in 3% w/v recombinant silk pre-gel solutions complemented with 1% w/v eADF4(k146)-coated FAp particles and gelled at 37° C. Cell viability was confirmed using trypan blue (Sigma-Aldrich, UK) and an automatic cell counter (TC20, Bio-Rad). Cells were live-dead stained with calcein acetoxymethyl ester (calcein AM) and ethidium homodimer I (Invitrogen, Thermo Fisher Scientific, Germany) for 45 min before imaging.

### 2. RESULTS

Here, a new set-up to process two separate materials out of one printer cartridge was established with the aim to generate scaffolds with gradient properties without strand breakup. Therefore, a set-up was numerically designed using computational fluid dynamics simulation. Afterwards, its usability was experimentally confirmed in a biofabrication application.

2.1 Flow Simulation of Gradient Material Generation during 3D Printing

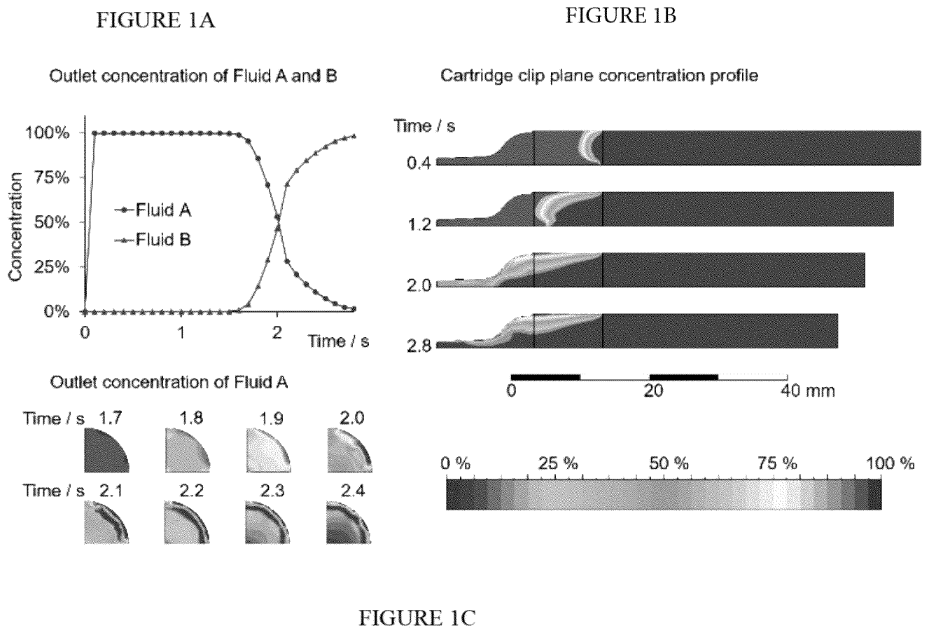

When two materials are present in one printer cartridge, a back-mixing effect is highly distinct both in flow simulations and in printing experiments (data not shown). This effect was related to the U-profile of a laminar material flow creating a velocity gradient. With the exemplary extrusion velocity of 5 mm/s, a back-mixing of two materials could be shown in the cartridge model, creating a gradient profile in the extruded strand. The concentration curve for both fluids at the cartridge outlet is shown in FIG. 1. When running the simulation, a core-shell effect of both materials was found whilst mixing, as no slip conditions were assumed at the cartridge walls. Furthermore, the residual material A at the cartridge walls was extruded after the piston came into contact with the piston crown.

### 2.2 Experimental Confirmation of Gradient Formation Using Dispense Plotting

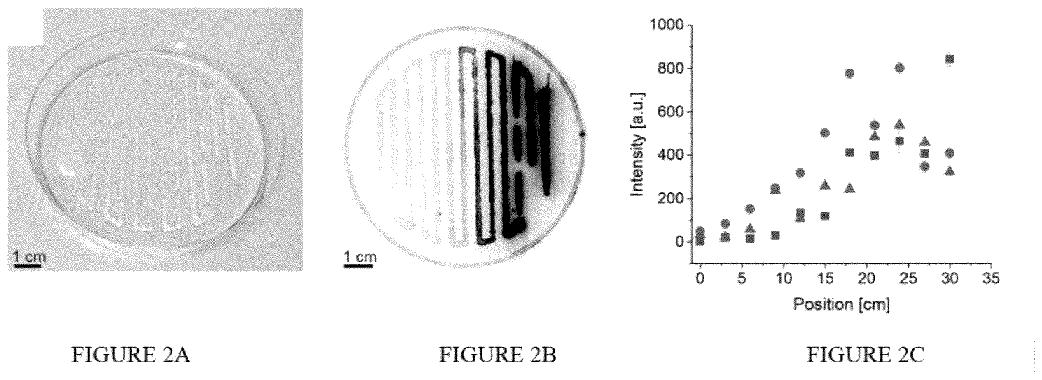

A commercial 3 cc printer cartridge was used providing a conical piston and a 20 G conical needle. A regenHU 3D Discovery printer was used with a printing speed of 20 mm/s and 0.1 bar. Two different materials, 0.5 mL each, were filled into one cartridge. First, coloured and uncoloured face cream were tested for visualization (data not shown). Then, hydrogels made of 3% w/v eADF4(C16) were prepared as one material block. In the second block, 10% w/w of fluorescently labelled FITC-eADF4 was added to the 3% w/v eADF4(C16) hydrogel. Only a slight decrease in viscosity and stiffness was observed in rheological measurements in case of the eADF4(C16)/FITC-eADF4(C16) blend hydrogel (data not shown), according to previous observations. A visual colourization from material B (eADF4(C16)/FITC-eADF4(C16)) at one side of the printed construct to a transparent hydrogel of material A (eADF4(C16)) was obtained after printing (FIG. 2a). Fluorescent imaging of the construct showed the gradual increase in fluorescence in accordance to the increase of FITC-eADF4(C16) content (FIG. 2b). Small amounts of the hydrogel were retracted from the construct at distinct locations for quantified fluorescence analysis. From unlabelled to fluorescence-labelled hydrogels, an increase in fluorescence intensity was measured using three independent scaffolds (FIG. 2c). This profile perfectly fitted the simulated material profile and confirmed the simulation to be in accordance with the experimental set-up.

The tendon-to-bone-interface shows a gradient in mechanical, compositional and structural cues, and, therefore, the applicability of the new system in biofabricating such interface was tested.

First, to mimic apatite mineralization towards the bone side, fluorapatite particles were gradually integrated.

### 2.3 Recombinant Silk Fluorapatite Composite Hydrogels and Bioactive Gradient Printing Thereof

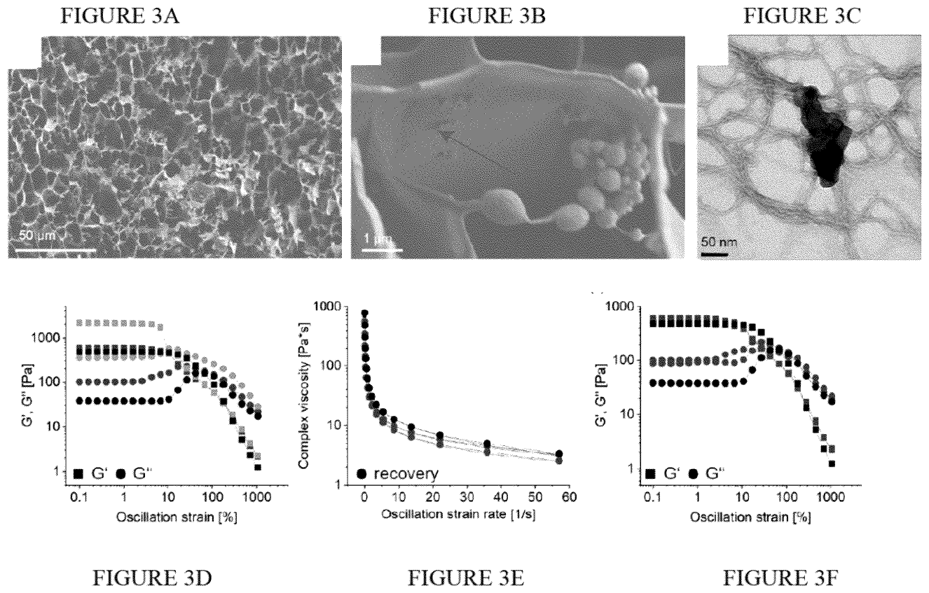

Fluorapatite (FAp) particles were synthesized using an ultrasonication approach from dry components, yielding rod-shaped FAp particles of about 92±27 nm length and 21±6 nm width as visualized using SEM and TEM (data not shown). The chemical integrity of the particles was identified in comparison to the quantitative occurrence of the elements Ca, F, O and P in FAp stoichiometry using SEM-EDX for element analysis (data not shown). The discrepancy between calculated and experimentally determined ratios varied for Ca:F and Ca:P ratios by 2%, for the Ca:O ratio by 21%. This is a result of a high content of O than expected, which could be explained by atmospheric water on the sample. Furthermore, ATR-FTIR spectra of FAp particles were compared to commercially available hydroxyapatite particles (data not shown). Both materials showed similar band assignments as previously reported for both apatite species[42]. The implementation of fluorapatite particles into recombinant silk solutions before gelation enabled an incorporation into the generated hydrogels as detected using SEM of freeze-dried hydrogels (FIG. 3a+b, arrow indicating free particles in a pore) and TEM which indicated intertwined FAp particles with eADF4(C16) fibrils (FIG. 3c). Concerning particle content, an increase from 1% w/v to 3% w/v yielded a higher initial storage modulus of the hydrogel (FIG. 3d). At higher strains, severe differences were visible as one material started to flow whilst the other was still static. For simultaneous printing of blank and particle-filled hydrogels from one cartridge it was crucial that both materials had flow points in the same order of magnitude. Therefore, a material blend of 3% w/v eADF4(C16) and 1% w/v FAp particles was used further on.

One important property of eADF4(C16) hydrogels is their shear thinning behaviour. In the presence of FAp particles, the hydrogels showed shear-thinning behaviour of a non-Newtonian fluid at increasing shear rates and recovery at decreasing shear rates, which is important to gain solid structures after strand deposition after 3D printing (FIG. 3e).

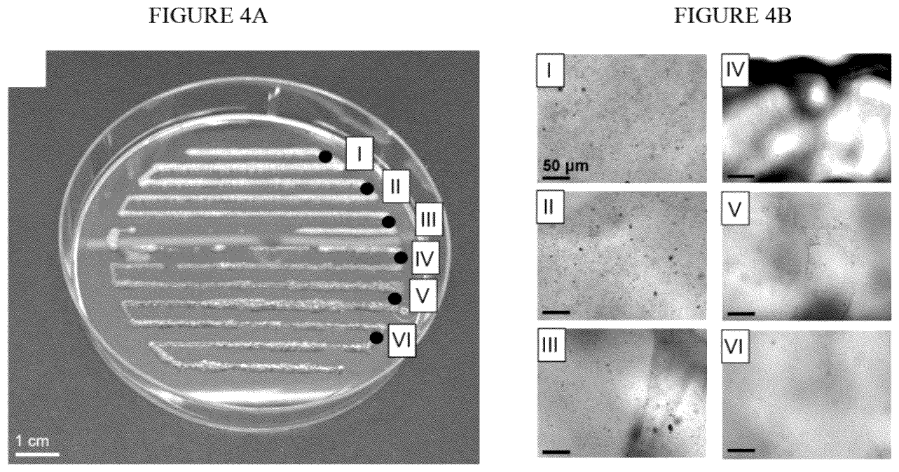

Gradient printing of recombinant silk hydrogels with FAp particles (material A) and without (material B) was realized (FIG. 4a) with the identical printing parameters as used for one-material hydrogels. There was a slight dewetting effect visible between position II and IV (FIG. 4a) without an influence on the general outcome. Light microscopy images at distinct positions revealed a decreasing particle density in the hydrogel (FIG. 4b, I-VI). Small particle aggregates in the lower micrometer range were visible, especially at regions with higher particle concentrations due to particle aggregation.

### 2.4 Biofabrication of Particle and Cell-Loaded Recombinant Silk Hydrogels

As FAp particles showed a negative zeta potential (−22.5±0.9 mV), the interaction of the particles with negatively charged eADF4(C16) in solutions could be increased by coating the FAp particles with the positively charged recombinant silk variant eADF4(κ16), referred to as kFAp (zeta potential+16.5±0.4 mV). Rheological behaviour was not influenced significantly when kFAp was incorporated into hydrogels (FIG. 3f). Further, eADF4(κ16) with its positive charge was found previously to enhance cell adhesion for biofabrication purposes.

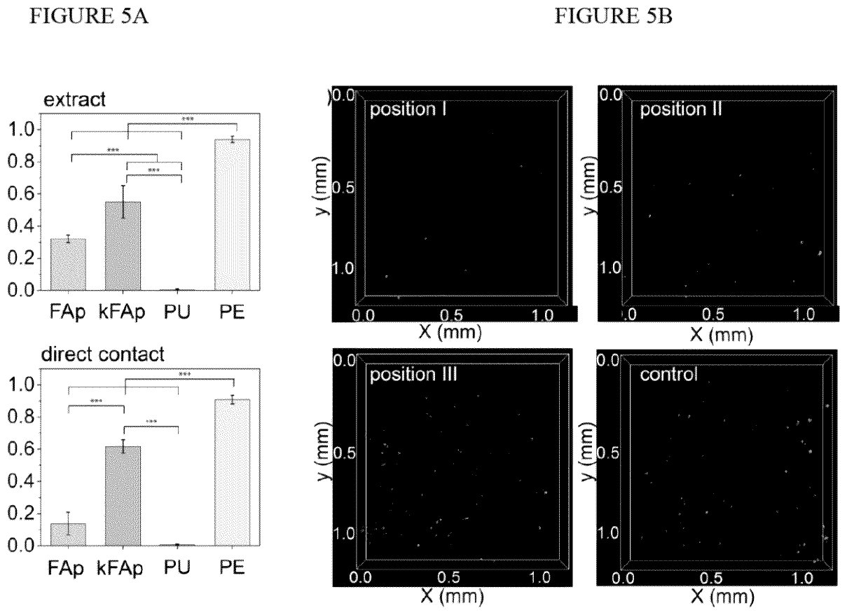

First, particles were tested regarding cell toxicity according to DIN EN ISO 10993-5. An extract test and a direct contact test were carried out. A cytotoxic effect is considered when cell viability is reduced by 30% referred to high density polyethylene used as positive control. As negative control, organotin-stabilized polyurethane was used. The quantification was normalized to tissue culture treated plate (TCP; as 100%). Fluorapatite particles were compared to eADF4(k16)-coated fluorapatite particles in both the extract and contact tests. In both cases, the protein coating enhanced the cell viability significantly (FIG. 5a). Cell toxic effects of apatite particles on cells were already reported in literature. Here, cell viability decreased in the presence of fluorapatite particles in the direct contact test to 13.8±7.1% and to 32±2% in the extract test. Particles sticked to the cells and could hardly be washed off (data not shown). In contrast, coated fluorapatite particles showed cell viability of 61.8±4% in the direct contact test and to 55.2 10% in the extract test.

For biofabrication, BALB/3T3 mouse fibroblasts were seeded at 106 cells/mL in 3% w/v eADF4(C16) hydrogels together with 1% w/v kFAp particles. The AB block system comprised 0.5 mL of 3% w/v eADF4(C16)+1% w/v kFAp+fibroblasts (A) and 3% w/v eADF4(C16) hydrogel (B). Printing was carried out as described above using a 16 G conical needle, as hydrogels with cells and particles were slightly stiffer as the non-cell-loaded ones. The obtained scaffolds were stained using calcein AM/ethidium homodimer I for live-dead evaluation of the cells. Recombinant silk hydrogels showed slight red autofluorescence. The confocal laser scanning microscopy images at distinct positions on the scaffold confirmed a successful gradient printing of BALB/3T3 fibroblasts (FIG. 5b). The simultaneous processing of apatite particles with a cell-friendly protein coating along with BALB/3T3 cells showed that this printing set-up offers not only the possibility to generate gradients from one printer cartridge, but also to incorporate multiple gradient features into one scaffold (here particles and cells), relevant for e.g. biofabrication of tissues for the tendon/bone interface.

### 3. CONCLUSIONS

Computational fluid dynamics combined with dispense plotting enabled to achieve an in situ generated gradient construct from one printer cartridge. This set-up could be applied for various material combinations and complexity levels, as mineralization gradients and further mineralization gradients along with cell gradients could be produced, especially useful for biofabrication applications. The combination of inorganic fillers and cells simultaneously in a material gradient can yield similar conditions as found at the enthesis, enabling future applications at the tendon-to bone interface and elsewhere.

1. A reservoir comprising
(i) a reservoir body defining an interior space of the reservoir, wherein a content is arranged in the interior space of the reservoir, and
(ii) an outlet,
wherein the reservoir is configured to dispense the content from the reservoir through the outlet by pressurising the content in the interior space of the reservoir, and
wherein the content comprises
a first flowable composition comprising a first material, and
a second flowable composition comprising a second material.

2-3. (canceled)

4. The reservoir of claim 1, wherein the reservoir is configured such that pressurisation of the content results in a non-plane boundary surface between the first flowable composition and the second flowable composition.

5. (canceled)

6. The reservoir of claim 4, wherein the non-plane boundary surface has a U-shaped cross section.

7. The reservoir of claim 1, wherein the reservoir is configured to dispense the content from the reservoir through the outlet with a laminar flow.

8. The reservoir of claim 1, wherein the reservoir is configured to dispense the content from the interior space through the outlet such that the dispensed content can exhibit a gradient or change in the proportion of the first flowable composition and the proportion of the second flowable composition in the dispensed content.

9-16. (canceled)

17. The reservoir of claim 1, wherein the first flowable composition and/or the second flowable composition comprise one or more additives.

18. The reservoir of claim 1, wherein the first material and the second material are identical or different from each other.

19-20. (canceled)

21. The reservoir of claim 18, wherein the difference between the first material and the second material is in the type, structure, concentration, and/or viscosity of the first material and the second material.

22-27. (canceled)

28. The reservoir of claim 1, wherein the first material is selected from the group consisting of a silk polypeptide, hyaluronic acid, silicone, collagen, alginate, gelatin, gelatin with methacrylated groups (GelMA), heparin, chondroitin sulfate, chitosan, and cellulose, preferably nanocellulose, and/or the second material is selected from the group consisting of a silk polypeptide, hyaluronic acid, silicone, collagen, alginate, gelatin, gelatin with methacrylated groups (GelMA), heparin, chondroitin sulfate, chitosan, and cellulose, preferably nanocellulose.

29. The reservoir of claim 28, wherein the first material comprised in the first flowable composition is a silk polypeptide and the second material comprised in the second flowable composition is a silk polypeptide, wherein the first flowable composition or the second flowable composition comprises one or more additives.

30-33. (canceled)

34. The reservoir of claim 1, wherein
the first flowable composition is a hydrogel, and/or
the second flowable composition is a hydrogel.

35. The reservoir of claim 1, wherein the reservoir is a cartridge, preferably a printer cartridge.

36. A printer comprising the cartridge of claim 35 or a receptacle for such a cartridge.

37. (canceled)

38. A method of dispensing a content from the reservoir of claim 1, comprising the step of:
applying pressure to a content of the reservoir, so that the content is dispensed from the reservoir via an outlet, wherein the content comprises
a first flowable composition comprising a first material, and
a second flowable composition comprising a second material.

39. The method of claim 38, wherein during dispensing content from the reservoir, at least a part of the dispensed content comprises at the outlet the first flowable composition and the second flowable composition which leave the reservoir simultaneously through the outlet.

40. The method of claim 39, wherein the first flowable composition and the second flowable composition which leave the reservoir simultaneously through the outlet are deposited simultaneously on a target surface.

41. The method of claim 38, wherein pressurisation of the content results in a non-plane boundary surface between the first flowable composition and second flowable composition.

42. The method of claim 41, wherein the boundary surface has a U-shaped cross section.

43-45. (canceled)

46. A kit comprising the reservoir of claim 1.

47-50. (canceled)

51. A method for bioprinting, biofabrication, tissue engineering applications, or gradient formation using the reservoir of claim 1.

52-56. (canceled)


SEQUENCE LISTING (8 sequences)

SEQ ID NO: 1 - protein, 35 aa, synthetic construct
  REGION 1..35: note = synthetic
  DOMAIN 1..35: note = Module C (ADF-4)
GSSAAAAAAAASGPGGYGPENQGPSGPGGYGPGGP

SEQ ID NO: 2 - protein, 35 aa, synthetic construct
  REGION 1..35: note = synthetic
  DOMAIN 1..35: note = Module Ccys
GSSAAAAAAAASGPGGYGPENQGPCGPGGYGPGGP

SEQ ID NO: 3 - protein, 35 aa, synthetic construct
  REGION 1..35: note = synthetic
  REGION 1..35: note = domain - Module Ck
GSSAAAAAAAASGPGGYGPKNQGPSGPGGYGPGGP

SEQ ID NO: 4 - protein, 35 aa, synthetic construct; identical to SEQ ID NO: 3
  REGION 1..35: note = synthetic
  REGION 1..35: note = Domain - Module Clys

SEQ ID NO: 5 - protein, 280 aa, synthetic construct
  REGION 1..280: note = synthetic
  REPEAT 1..280: note = C8
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 6 - protein, 560 aa, synthetic construct
  REGION 1..560: note = synthetic
  REPEAT 1..560: note = C16
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 7 - protein, 1120 aa, synthetic construct
  REGION 1..1120: note = synthetic
  REPEAT 1..1120: note = C32
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 8 - protein, 1680 aa, synthetic construct
  REGION 1..1680: note = synthetic
  REPEAT 1..1680: note = C48
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP